(19) United States
(12) Patent Application Publication
(10) Pub. No.: US 2022/0409878 A1
(43) Pub. Date: Dec. 29, 2022

(54) BLOOD PUMP SYSTEM WITH MAGNETICALLY LEVITATED ROTOR

(71) Applicant: Berlin Heart GmbH, Berlin (DE)

(72) Inventors: Oliver PETERS, Berlin (DE); Valentin BYKOV, Berlin (DE); Hendryk RICHERT, Berlin (DE); Michael FRISCHKE, Rangsdorf (DE)

(73) Assignee: Berlin Heart GmbH, Berlin (DE)

(21) Appl. No.: 17/780,203

(22) PCT Filed: Nov. 27, 2020
(86) PCT No.: PCT/EP2020/083729

§ 371 Date: May 26, 2022

(30) Foreign Application Priority Data
Nov. 27, 2019 (EP) 19211940.2

(51) Int. Cl. A61M 60/178; A61M 60/226; A61M 60/546; A61M 60/422; A61M 60/822; A61M 60/859; A61M 60/863

(52) U.S. Cl. CPC A61M 60/178; A61M 60/226; A61M 60/546; A61M 60/422; A61M 60/822; A61M 60/859; A61M 60/863

(57) ABSTRACT

The application pertains to a blood pump system, in particular a ventricular assist device, VAD, the system including a blood pump, which comprises: a housing, including an inlet and an outlet, preferably an axial influx and a tangential outflow; a motor actuator, wherein the motor includes a plurality of motor coils (for driving an impeller); an impeller, wherein the impeller is located in the housing and includes a plurality of rotor magnets.

The system further comprises a drive line; and a control unit for controlling operation of the pump, the control unit configured to:
operate the motor, such that the impeller rotates around an axis; and
measure the rotor position in a direction along the axis using at least one of the plurality of the motor coils.

### CROSS-REFERENCE TO RELATED APPLICATIONS

This application is a 371 nationalization of international patent application PCT/EP2020/083729 filed Nov. 27, 2020, which claims priority under 35 USC § 119 to European patent application EP 19 211 940.2 filed Nov. 27, 2019. The entire contents of each of the above-identified applications are hereby incorporated by reference.

### BRIEF DESCRIPTION OF THE DRAWINGS

FIG. 1 shows a concept of a magnetically levitated ventricular assist device;

FIG. 2 shows a blood pump in which two axial flux motors are used to compensate reluctance forces (Source: US 2016/0281728A1);

FIG. 3 shows a VAD with a radial motor which balances the radial reluctance forces between rotor and stator (Source: U.S. Pat. No. 5,588,812A);

FIG. 4 shows a pump with compensation of reluctance forces by using a magnetic compensation bearing (Source: US2016/0281728A1);

FIG. 5 shows a VAD with ironless axial flux motor (Source: US006071093A)

FIG. 6 shows principles of simultaneous axial force and torque generation with an axial flux motor (Source: US006071093A);

FIG. 7 shows typical current and voltage waveforms for the self-sensing operation according to U.S. Pat. No. 6,302,6611B1;

FIG. 8 shows a block diagram of the rotor position estimation as disclosed in U.S. Pat. No. 6,302,661B1;

FIG. 9 shows a block diagram of a VAD with sensorless magnetic levitation;

FIG. 10 shows a block diagram of a VAD with sensorless magnetic levitation with additional components that are improving system redundancy and rotor position measurement signal-to-noise ratio;

FIG. 11 shows an electrical equivalent circuit of a direct current motor;

FIG. 12a shows an electrical equivalent circuit of a brushless direct current motor;

FIG. 12b shows an electrical equivalent circuit of a brushless direct current motor with a capacitor parallel to resistance and inductance of a motor coil in each motor phase;

FIG. 13 shows the electrical and mathematical structure of state of the art electromotive force measurements systems as used for sensorless commutation of BLDC motors;

FIG. 14 shows the method of using an inductive shunt to estimate the BEMF;

FIG. 15 shows an analogue BEMF replication

FIG. 16 shows a sampling of electric current using a combined resistive and inductive shunt;

FIG. 17 shows a method to emulate an inductive shunt with an inductive shunt and filtering;

FIG. 18 shows a filter structure used to generate magnetic field strength estimation from the BEMF estimation that uses a matched pair of a high pass and a low pass filter;

FIG. 19 shows an arrangement of motor coils that can be used to measure an axial and/or rotational position of the rotor with eddy current measurements using the motor coils;

FIG. 20 shows an equivalent circuit diagram of a motor coil with eddy current measurement and capacitive tuning;

FIG. 21 shows various implementations of a magnetically sensitive tuning capacitor;

FIG. 22 shows how a tuning network can be used to modify the resonant frequency of the motor coil with dedicated sensor elements with voltage signal output;

FIG. 23 shows voltage and current waveforms in motor drivers with and without power filters;

FIG. 24 shows energy flow and spectrum on the driveline when connecting a RF source to the driveline wires;

FIG. 25 shows energy flow and spectrum for a controller and pump system with integrated RF motor coil impedance measurement and RF filters;

FIG. 26 shows a block diagram of noise filters in the current measurements chain;

FIG. 27 shows a block diagram of a class AB motor driver with a dynamic power supply;

FIG. 28 shows an AC-inverter motor controller;

FIG. 29 shows a spectrum of motor driver PWM signal and RF impedance measurement without influence of jitter;

FIG. 30 shows an influence of clock jitter on the PWM spectrum and the RF impedance measurement spectrum;

FIG. 31 shows a block diagram of a motor driver and impedance measurement with shared clock source;

FIG. 32 shows an electrical structure of a power connector with additional sense contacts;

FIG. 33 illustrates a motor connection scheme that uses a fourth driveline wire to increase VAD system redundancy;

FIG. 34 illustrates a motor connection scheme that connects a dedicated bearing coil to the star point and a fourth driveline wire;

FIG. 35 illustrates a motor connection scheme with six driveline wires, a motor and a dedicated bearing coil where every driveline can fail with an open connection and pump operation is still possible;

FIG. 36 illustrates a motor connection scheme with four driveline wires, a motor and a dedicated bearing coil without fail safe features;

FIG. 37 illustrates a motor connection scheme with seven driveline wires, a motor and a dedicated bearing coil is which even after a driveline open connection failure the pump can be operated without reduced motor efficiency;

FIG. 38 shows a cross-section of an embodiment of a blood pump to be used as a VAD that uses a combined rotational and linear motor;

FIGS. 39a and 39b show a) a cross-section of an embodiment of a blood pump to be used as a VAD according to this document and a b) top view of a section of the rotor of the blood pump;

FIG. 40 shows a cross-section of an embodiment of a blood pump to be used as a VAD according to the present disclosure;

FIG. 41 shows a cross-section of an embodiment of a blood pump to be used as a VAD according to the present disclosure;

FIG. 42 shows a cross-section of an embodiment of a blood pump to be used as a VAD according to the present disclosure;

FIG. 43 shows a cross-section of an embodiment of a blood pump to be used as a VAD according to the present disclosure;

FIG. 44 shows a cross-section of an embodiment of a blood pump to be used as a VAD according to the present disclosure;

FIG. 45 shows a systematic of factor tuning in the context of BEMF-based rotor position estimation methods;

FIG. 46a shows how an integration error is generated, when integrating the BEMF to obtain the B-field;

FIG. 46b shows a low-pass filter in a self-regulating servo-loop;

FIG. 46c shows a disadvantage of a servo-loop and how a moving-average filter may remedy this disadvantage;

FIG. 47a shows a diagram depicting a frequency versus impedance curve of a series connection of resonant circuits, wherein a resonant circuit may be used as an eddy current sensor to measure a rotor position, for example a rotor angle;

FIG. 47b shows, similar to FIG. 47a, the impedance curve, wherein in one of the resonant circuits there is an additional capacitor in parallel to a parasitic capacitance;

FIG. 47c shows, similar to FIG. 47b, the impedance curve, wherein there is an additional capacitor in two of the resonant circuits and the capacitors each have a different capacitance;

FIG. 47d shows, similar to FIG. 47c, the impedance curve wherein the rotor assumes a tilted position.

FIG. 48 shows a cross-section of an embodiment of a blood pump to be used as a VAD according to the present disclosure;

FIG. 49 shows a cross-section of an embodiment of a blood pump to be used as a VAD according to the present disclosure;

FIG. 50 shows a cross-section of an embodiment of a blood pump to be used as a VAD according to the present disclosure;

FIG. 51 shows a cross-section of an embodiment of a blood pump to be used as a VAD according to the present disclosure;

FIG. 52 shows a cross-section of an embodiment of a blood pump to be used as a VAD according to the present disclosure;

FIG. 53a shows schematically an axial cross-sectional view of an end of a cannula of a connection system before joining;

FIG. 53b shows schematically an axial cross-sectional view of a tube end of the connection system before joining;

FIG. 53c shows schematically an axial cross-sectional view of the joint between the cannula end and the tube end of the connection system;

FIG. 54 shows a perspective view of a claw ring of the connection system;

FIG. 55a shows a perspective view of a locking ring as viewed towards the tube end of the connection system;

FIG. 55b shows a perspective view of a locking ring as viewed towards the cannula end of the connection system;

FIG. 56a shows an axial cross-sectional view of a claw ring of the connection system;

FIG. 56b shows an axial cross-sectional view of a locking ring of the connection system;

FIG. 56c shows an axial cross-sectional view of a claw ring and locking ring in a latched state of the connection system

FIG. 57a shows an arrangement of the claws when pushed onto the locking ring (latching position) of the connection system;

FIG. 57b shows an arrangement of the claws on the locking ring after a rotation of the claw ring in an unlatched position, wherein one claw is not spread open and contacts the rotation limit stop, thereby defining the unlatching position;

FIG. 58a shows a cannula of a device for connecting a cannula;

FIG. 58b shows a suture ring of the device for connecting a cannula;

FIG. 58c shows the suture ring of the device for connecting a cannula sutured to a hollow organ.

### DETAILED DESCRIPTION

The ventricular assist market offers currently no good implantable heart assist solution for children with a body surface area below 1.3 m². Large ventricular assist devices (VAD), intended and approved for the use in adults, are sometimes used in children due to the lack of a more suitable implantable VAD. The outcome of this off-label use of adult VADs in children is significantly worse than the approved therapy in adults.

To adapt a VAD for children, the size and fluidic performance have to be optimized without compromising hemocompatibility.

Blood pumps having a magnetically levitated rotor are currently regarded as state of the art to implement a hemocompatible blood pump. However, the miniaturization of a magnetic bearing is limited by its complexity and number of components.

It is an objective of the presented invention to provide an improved VAD including a blood pump suitable for being applied to or within children. In particular, a novel solution is provided to reduce the complexity and number of components in a blood pump having a magnetically supported rotor. The present disclosure also concerns aspects of system safety, operational system safety and system redundancy of a blood pump as well as aspects of connecting the blood pump to a blood circulation.

Exemplary embodiments are shown in the attached figures.

The pumps, motor drivers and methods described herein allow operation of a motor for driving the rotor, measuring the rotor position using the motor and influencing the rotor position.

To use the motor for driving the rotor and for measuring the rotor position in at least one degree of freedom (DOF), such as, for example, along a direction of the axis of rotation, several embodiments and improvements over the known prior art are presented.

The disclosed blood pump system, in particular a ventricular assist device, VAD, system includes a blood pump, the pump comprising a housing, including an inlet and an outlet, preferably an axial influx and a tangential outflow, a motor actuator, wherein the motor includes a plurality of motor coils (for driving an impeller) and a rotor including the impeller, wherein the impeller is located in the housing and includes a plurality of rotor magnets. Furthermore, the system comprises a drive line, a control unit for controlling operation of the pump, the control unit configured to operate the motor, such that the impeller rotates around an axis, and measure the rotor position in a direction along the axis using at least one of the plurality of the motor coils.

Rotor and impeller are rigidly connected with each other. They may also be integrally connected.

A motor coil is configured to provide a magnetic field used to apply torque to the rotor. A motor coil may also be considered as an actuator coil. Coils of an active magnetic bearing may also be considered as an actuator coil. It is possible that a motor coil is used as a coil of an active magnetic bearing.

In an embodiment it is possible that the control unit is configured to reduce or eliminate switching noise from a motor driver. The motor driver may generate a pulse width modulated (PWM) signal to control the motor coils. This PWM signal is generated by switching switches, for example transistors may be used for switching, having switching noise as a side effect. The switching noise, which includes high frequency electric current and voltage components, in particular harmonics of the switching frequency, may be reduced, for example, by the application of a power filter which is a low pass configured to suppress harmonic components in the switched signal. Another method to reduce switching noise may be to not switch at all, for example by using a class AB amplifier and it is also possible to apply a tracking DC-DC controller having a smoother output than a PWM based motor driver.

Optionally it is also possible that an output stage of the motor driver includes filter elements for filtering out high frequency signal components. As an example it is possible to use filter elements having a low pass characteristic. However, in order to remove harmonics from the motor driver signal it is as well possible to use a band stop filter or a band pass filter. It may also be possible to use filters with a plurality of stop bands. It may also be considered to use filters with a plurality of pass bands.

It may be considered to add a high frequency signal to the filtered motor driver output. The high frequency signal may, for example, be used to determine an impedance of one or more of the motor coils. It is hence possible that a measurement of the motor currents includes a measurement of the motor coil impedance, preferably the high frequency motor coil impedance. The high frequency signal may have a frequency greater than the rotational frequency of the rotor. It is conceivable that the high frequency signal has, for example, a frequency greater than 1 MHz or 3 MHz, but may also have a frequency between 100 kHz and 1 MHz, for example between 100 kHz and 300 kHz. It is possible that the high frequency signal has a frequency not coinciding with a harmonic frequency of the motor driver output signal. It is furthermore possible that the high frequency signal is close or equal to a resonance frequency of a motor coil. The resonance frequency of a motor coil may originate from the inductance of the motor coil and a parasitic or stray capacity of the motor coil. The capacitance of the motor coil may be adjusted by, for example, parallel connecting a capacitor.

In an embodiment it is also possible to replicate a motor internal back-electromotive force (BEMF) outside the motor, for example using an inductive shunt voltage measurement. Based on the physical induction law, the BEMF is generated by moving permanent magnets that may be fixed on the rotor, close to a motor coil. The BEMF is then generated within windings of the motor coil. A replication, that is an estimation, of the BEMF generated within the windings of the motor coil, may be possible if the inductance of the motor coil and the resistance of the motor coil are known. Then, by obtaining the value of the electric current through the coil by means of a measurement an estimate of the BEMF is possible. The accuracy of the estimate may depend on the accuracy of the impedance and resistor values used in the calculation. For the measurement of the electric current in the motor coil, a shunt resistor may be used; it is also possible that a shunt inductance is used.

It is furthermore conceivable that a magnetic field strength is replicated in an electrical or digital signal or also a combination thereof outside the motor, preferably using a BEMF replica and a matched pair of high-pass and low-pass filter elements. As an example the low pass and the high pass may be complementary filters whose transfer functions add up to a constant value. By processing the BEMF signal separately in a high pass and low pass signal flow path the accuracy of a position estimate, for example a position estimate in axial direction of the rotor, may be improved. The axial direction is a direction parallel to the main rotational axis of the rotor virtually connecting an axial centre of an inlet and a centre of a back plate of the blood pump.

In certain embodiments the control unit may reduce voltage transients in the driveline. It is possible that the control unit is configured to reduce trapezoidal or triangular current waveforms with respect to the sinusoidal current waveforms.

As an option the control unit may include a DC-DC converter. The control unit may include one or more class AB amplifiers. It is as well conceivable that the control unit comprises passive filter elements. This option may be used to reduce and/or attenuate the harmonics in the driveline signal and hence to reduce high frequency switching noise which may, for example, contaminate a position measurement. The class AB amplifier may be used to amplify a driveline signal generated by the control unit and having more than two amplitude levels as in a class D type amplifier. The increased number of amplitude levels of the driveline signal may significantly reduce the generation of harmonics and switching noise. However, the efficiency of a class AB amplifier depends on the level of the supply voltage with respect to signal amplitude. For this reason an adjustable DC-DC converter may be used to adjust the supply voltage of the class AB amplifier and hence to improve the efficiency thereof. Remaining amplitude steps after amplification of the driveline signal may be smoothened with a passive filter, for example a low pass filter. The passive filtering may be carried out before or after amplification of the driveline signal.

In an embodiment the driveline may include no more than four wires, preferably three wires and one redundant wire.

The blood pump may include a passive magnetic radial bearing, wherein a passive magnetic bearing supports a rotor with one or more permanent magnets. Optionally the blood pump may include a passive magnetic tilt bearing. In certain embodiments the blood pump may include an active axial magnetic bearing configured to actively control a rotor position with respect to an axial degree of freedom. An active magnetic bearing supports a rotor with an electromagnetic force wherein the electromagnetic force may be adjusted by closed loop control. According to Earnshaw's theorem it is not possible to support a rotor exclusively with passive magnetic bearings. It is also conceivable that another degree of freedom than the axial is actively controlled. In this case, the axial degree of freedom may be supported with passive magnetic bearings.

It is possible that the electric motor is, for example, a brushless direct current (BLDC) motor. It is possible that other types of motors are used such as a synchronous motor or an induction motor or other types of motors such as a DC motor. It is also conceivable that the motor is an axial flux motor, preferably an ironless axial flux motor.

The motor of the blood pump system may include a capacitor electrically parallel connected to a motor coil, wherein the motor coil and the capacitor form a resonant circuit having a resonance frequency and an electrical impedance with a magnitude and a phase. In a high frequency equivalent circuit the motor coil may be considered as a series connection of an inductance and a resistor and a parasitic or stray capacitance in parallel to said series connection of resistor and inductance. The capacitances of the capacitor and the stray capacitance add up. Capacitance and inductance form a resonant circuit having a resonance frequency. The resistor introduces damping to said resonant circuit. With the additional capacitor the resonance frequency of the resonant circuit may be adjusted.

In addition, the motor coil may include a first coil and a first capacitor may be electrically parallel connected to the first coil and both forming a first resonant circuit. Furthermore, the motor coil may include a second coil and a second capacitor may be electrically parallel connected to the second coil and both forming a second resonant circuit. It is possible that a capacitance of the first capacitor is different from a capacitance of the second capacitor and the resonance frequency of the first resonant circuit is different from the resonance frequency of the second resonant circuit. By having different capacitances of the first capacitor and the second capacitor it is possible to achieve different resonance frequencies in the resonant circuits of the different motor coils arranged in different motor phases. This may be used to allocate a resonance to a motor phase or motor coil and, based thereon, to determine a position of the rotor, for example with respect to a spatial coordinate system fixed to a motor stator.

The blood pump system may further include a measurement unit configured to determine the electrical impedance of one or more of the resonant circuits. The electrical impedance is a quantity that may depend on the rotor position. The combined impedance of two motor coils may be determined from a measurement at the corresponding phase terminals of the motor. The contribution of each motor coil impedance to the combined impedance may be determined if the parallel connected capacitors of the motor coils have different values for each motor coil such that the resonance frequencies of the resonant circuit in each motor phase are different.

It is conceivable that the blood pump system includes an estimation unit configured to estimate a translational and/or a rotational position of the rotor based on the electrical impedance of one or more of the resonant circuits. For example, when using different materials along the perimeter of the rotor, the impedance of a motor coil varies with the angular position of the rotor, that is, the resonance frequency of a motor coil varies with the angular position of the rotor. By evaluating the impedances close to a reference, for example average, resonance frequency for each motor coil it is hence possible to determine the angular position of the rotor by analysing the impedance variation for one or more of the motor coils.

All measurements that are influenced by the electrical impedance of the motor coil or its resonant behaviour are also considered as electrical impedance measurements. For example, by using the resonant circuit as frequency selecting element in an oscillator circuit, the resonance frequency can be measured without measuring the impedance directly. However, the frequency behaviour is completely described by the resonators impedance characteristic. Other examples for indirect electrical impedance measurements include resonant decay measurements, oscillator quality measurements or phase shift measurements.

As an option, a test signal may be fed into a motor coil, wherein the test signal may include a component which is at least one of amplitude modulated, frequency modulated, phase modulated, code modulated, wherein the code modulated component preferably includes a random code modulated component or a pseudo random code modulated component. Using the modulation, the test signal may be detected in a more robust manner. Also as an option, the blood pump system may further comprise a detector unit, preferably including a correlator or a synchronous detector, configured to detect the test signal in a voltage measured across the motor coil and/or in a signal derived thereof, for example the BEMF. In an embodiment, the detector unit is configured to estimate the motor coil impedance based on the detected test signal. For example, based on a measurement of the voltage across a motor coil and a measurement of the current within the coil, for example using a shunt resistor, and by using an estimated motor coil impedance comprising an estimated resistance and an estimated inductance, it is possible to determine the BEMF generated within the coil by estimating the voltage across the estimated motor coil impedance and subtracting the estimated voltage from the voltage measured across the coil. If the estimated motor coil impedance is accurate, the modulated test signal vanishes from the estimated BEMF. Otherwise, the modulated test signal is still detectable, by the detector unit, within the estimated BEMF.

Also in an embodiment, the motor coil impedance may continuously be estimated during operation of the blood pump system. It is a further possibility that the BEMF replica is calculated with the estimated motor coil impedance and it is possible that the BEMF replica is continuously calculated.

As a conceivable option, the estimated motor coil impedance may be estimated by minimizing the test signal component within the BEMF replica.

The amplitude of the detected test signal may be used to adjust the estimated motor coil impedance, for example by minimizing the amplitude of the detected test signal with respect to the estimated motor coil impedance. The minimization of the test component may be accomplished with a numeric minimization or optimization procedure, for example a gradient based algorithm or the like, or a control algorithm like a PI- or an I-controller or the like.

As a possibility, a magnetic field strength may be replicated in an electrical or digital signal outside the motor, preferably by integrating the BEMF replica with an integrator, wherein the integrator is numerically stabilized by feeding back an output signal of the integrator via a moving average filter, which produces an averaged signal, to an input of the integrator. Stabilization of the integrator means to prevent the output signal of the integrator from drifting, that is, to bring or to keep a mean value of the integrator output signal to zero.

By feeding back the average of the integrator output to the integrator input it is possible to prevent, in a stationary case, a non-zero mean value of the integrator output signal. So, in an embodiment the BEMF replica may be an input signal of the integrator and the averaged signal may be subtracted from the input signal of the integrator. In certain embodiments it may be advantageous that the averaging time of the moving average filter is one rotation period or an integer multiple of one rotation period of the rotor. It is also possible that the averaged signal is low pass filtered before being subtracted from the input signal of the integrator.

The blood pump system further may also include a connection system for use in medical applications comprising:
a cannula made of a flexible material, a claw ring disposed on the cannula and having at least two claws, wherein the claw ring encompasses an outer surface of the cannula and is arranged on a cannula end of the cannula for rotation and axial displacement on the cannula to a stop, the stop comprising a collar on the cannula end on the outer surface of the cannula, and
a tube comprising a locking ring attached to a tube end and a nipple attached to the tube, wherein the claw ring is capable to be joined with the locking ring by an axial movement of the claw ring with respect to the cannula towards the locking ring and by latching of the at least two claws on the locking ring in a position in which this axial movement is limited by the stop.

As advantages of the connection system one may consider the following:
It is possible to implement a simple, quicker and safe connection of a flexible hollow tube with a metal tube under implantation conditions for a blood pump, wherein this connection can be released by rotating the claw ring and pulling the claw ring off the cannula in an axial direction.
The outside diameter at the connection location between the cannula and the tube increases by only a small amount relative to the outside diameter of the cannula or of the tube, respectively, which reduces the weight.
The connection conditions are reproducible.
The axial retention and the radial and/or axial seal are separate, unlike with connections using a union nut.
The claw ring guarantees axial retention and prevents the connection from separating. The radial and/or axial seal is achieved by optimizing the diameter ratio and/or thickness ratio at the nipple (hose coupling) and at the cannula. As a result, no undefined axial or radial force is applied to the cannula, i.e. the material of the cannula is not adversely affected. Also eliminated is an additional rotation lock, since the claw ring would have to be rotated for releasing the connection, which would require a torque that can spread the claws by sliding of the sloped faces of the locking ring. If necessary, this torque can be supplied by the operator. Accordingly, this represents a self-locking arrangement.
With the snap connection, an ideal and gentle transition from the cannula to the tube can be achieved. The connection between the nipple in the form of a hose coupling, which is typically made of titanium, and the cannula have practically zero flow resistance.
The connection system can operate with any known cannula material and does not require substantial design changes. No special tool is required for coupling and decoupling the device according to the connection system.
The length of the outlet cannula is adapted at the pump end by cutting off unnecessary reinforcement elements. The claw ring is then again pushed on the cannula and the spacer element is inserted into the groove closest to the cannula end. The outlet end of the cannula can then be designed without restraint.

In addition, the blood pump system may include a device for connecting a cannula with a hollow organ, in particular with a heart, characterized in that a cannula tip of the cannula has an opening which, for the prevention of complete occlusion and retention of blood flow from the hollow organ into the cannula, is waved at its upper edge and provided with recesses.

In an embodiment, the cannula may be combined with a suture ring suturable at the heart. It is conceivable that the cannula has a suture flange.

With the cannula proposed here, a jet flow and thus the frequency of an occurrence of thromboatheroembolism is reduced. Compared to a rigid design the device for connecting a cannula with a hollow organ has two particular advantages: Because the apex of the heart makes both lateral and rotational movements during the cardiac cycle (due to the helical arrangement of cardiac muscle fibres), the flexible cannula can absorb these movements and thus prevent the development of forces acting on the interfaces of the heart muscle. These forces are potentially dangerous since they may lead to bleeding or heart muscle damage. Furthermore, due to a flexible elbow of the device, the surgeon has the opportunity to adapt the position of the blood pump to the anatomical features.

Turning now to the drawings. The drawings described herein illustrate embodiments of the presently disclosed subject matter, and are illustrative of selected principles and teachings of the present disclosure. However, the drawings do not illustrate all possible implementations of the presently disclosed subject matter, and are not intended to limit the scope of the present disclosure in any way.

To levitate a rotor, all forces acting on the rotor have to be counteracted by equal opposing forces. A common method uses passive magnets to create counter-forces when the rotor is displaced.

According to Earnshaw's theorem, not all degrees of freedom can be stabilized at the same time using passive magnets. An arrangement of permanent magnets that stabilizes a certain axis will always destabilize another axis by at least the same amount.

Earnshaw's theorem also states that the sum of all stabilities and instabilities is always zero for arrangements of passive magnets. The common approach is to stabilize some axis using magnets and create some unstable degrees of freedom (DOF) which have points of unstable equilibria. The DOF with an unstable equilibrium are then actively controlled. A common control strategy is zero force control which balances the DOF at the unstable equilibrium to minimize the necessary power for levitation.

At the zero force position or unstable equilibrium, the power consumption can approach zero and is only limited by the noise and time delays in the control loop. Common for implantable blood pumps are power consumptions of 500 mW (e.g. Berlin Heart Incor) for the levitation control.

When the zero force control is not in operation, then the rotor leaves the equilibrium point and is accelerated until it makes contact with the pump housing. The force acting on the rotor when it is resting on the pump housing is equal to the maximum force the rotor can tolerate under zero force control.

This force is also equal to the force necessary to detach the rotor from the pump housing. Therefore, lowering the detachment force is only possible when also reducing the maximum tolerable load on the rotor. The peak detachment power can be orders of magnitude higher than the zero force levitation power. In the INCOR VAD it is three orders magnitude (0.5 W for levitation and 500 W for detachment) and in the Heart Mate 3 VAD it is roughly two orders of magnitude (0.5 W for levitation and 50 W for detachment). Reducing the detachment forces is one way of reducing the detachment power.

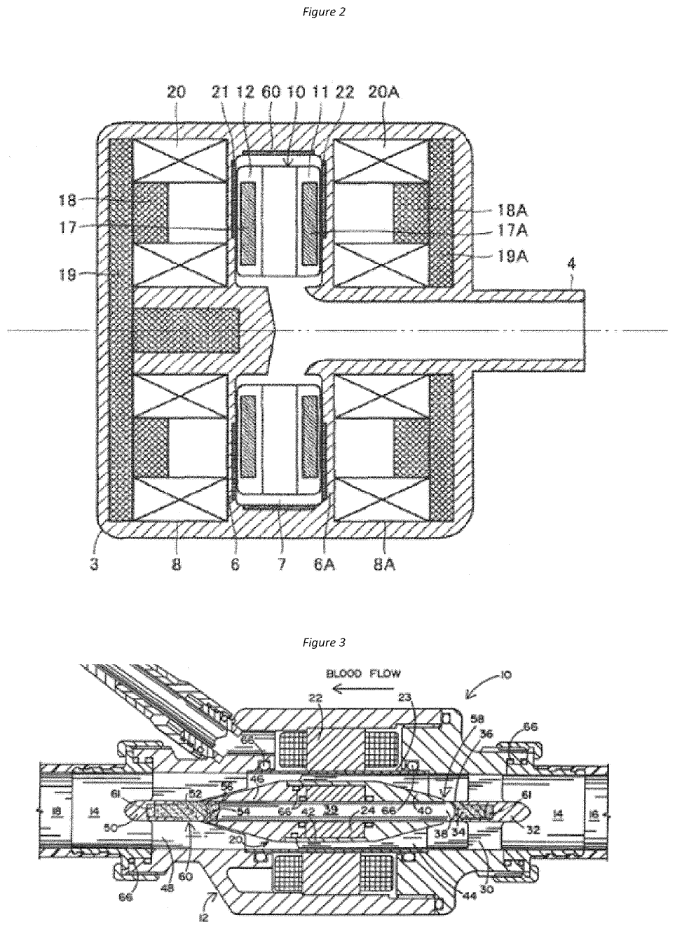

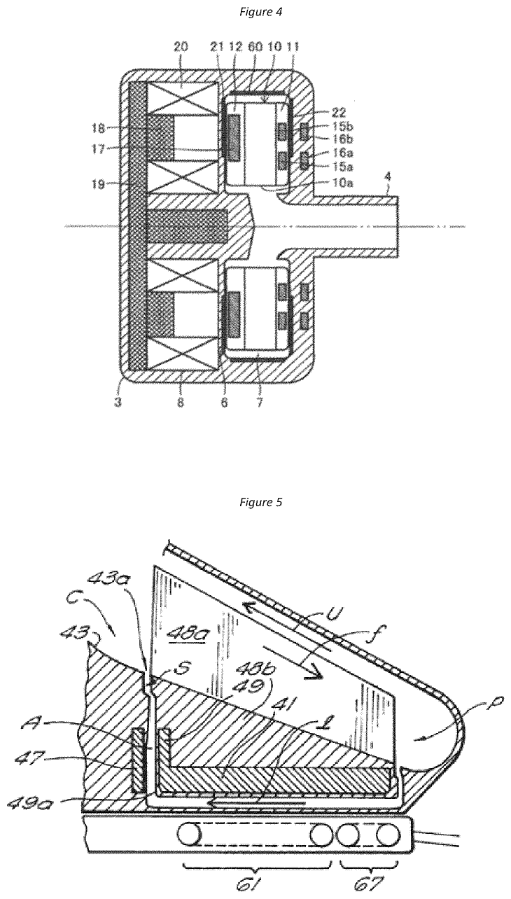

The force between rotor magnets and motor stator, usually reluctance forces, can also contribute significantly to the load on the rotor as well as the detachment forces. Most brushless direct current (BLDC) motors have a magnetic rotor and iron in the stator. This leads to constant attraction forces between rotor and the stator. This load force can be reduced by using two stator components whose forces on the rotor are cancelling each other out, as can be seen in the stator configuration in FIG. 2 (US20160281728A1) and FIG. 4 (US20160281728A1.) Another option to balance the static reluctance forces is to use a radial flux motor like it can be found in the Heart Mate 2 VAD (U.S. Pat. No. 5,588,812A, FIG. 3). However, while the load force can be balanced, the magnetic stiffness is always influenced negatively by introducing more iron as part of the motor stators. This leads to disturbance forces when the rotor is not perfectly balanced at its equilibrium position.

The reluctance load force can be eliminated completely by using an ironless stator configuration. The ironless stator also has zero influence on the magnetic suspension stiffness of the rotor.

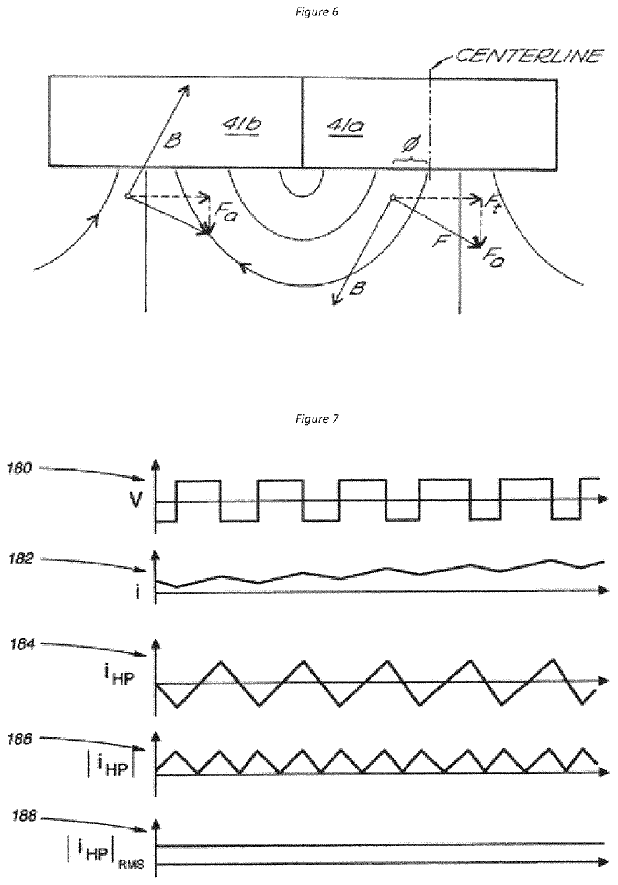

An ironless axial flux disc motor can generate torque as well as positive and negative pull force. The patent document U.S. Pat. No. 6,071,093A (FIG. 5, FIG. 6) shows a radial blood flow pump with an ironless axial flux motor where the motor coils are used for axial thrust generation and torque generation. The ironless (coreless and yokeless) axial flux motor is so far not used in a VAD system. The reason is probably the reduced motor efficiency of an ironless motor compared to a motor with iron yoke or cores. However, in an actively levitated VAD system the drawback of lower efficiency opposes the gain in levitation stability. As long as the motor can be cooled sufficiently without warming the blood too much, the ironless or iron reduced axial flux motor can lead to a smaller VAD device because a yoke or magnetic compensation bearing are no longer necessary. A thermal management which implements the features presented in the patent application EP19159286.4 can make the ironless axial flux motor feasible for use in an implantable VAD.

The pump presented in the patent document U.S. Pat. No. 6,071,093A uses dedicated sensors to detect the axial rotor position. These sensors have to be read out by additional driveline wires or an electronic system which is integrated into the pump. Both solution increase the size of the implantable VAD system and therefore reduce the viability for a paediatric VAD system.

Sensorless operation of BLDC motors is commonly used in VAD systems. Sensorless BLDC operation refers commonly to a sensorless commutation of the motor phases by measuring the induced back electromotive force (BEMF) or estimating the BEMF by measuring the current in the motor phases and estimating the BEMF based on the current waveforms. The BEMF signal is proportional to rotor speed. This implies that the signal is low at low rotational speeds and zero at standstill. Below a certain speed the noise of the current or voltage measurement compromises the BEMF detection and sensorless BEMF commutation is not possible.

For commutation and motor operation only the rotational position of the rotor has to be known and measured. For magnetic levitation the position of the rotor in the unstable DOF, which is usually a linear DOF, has to be measured.

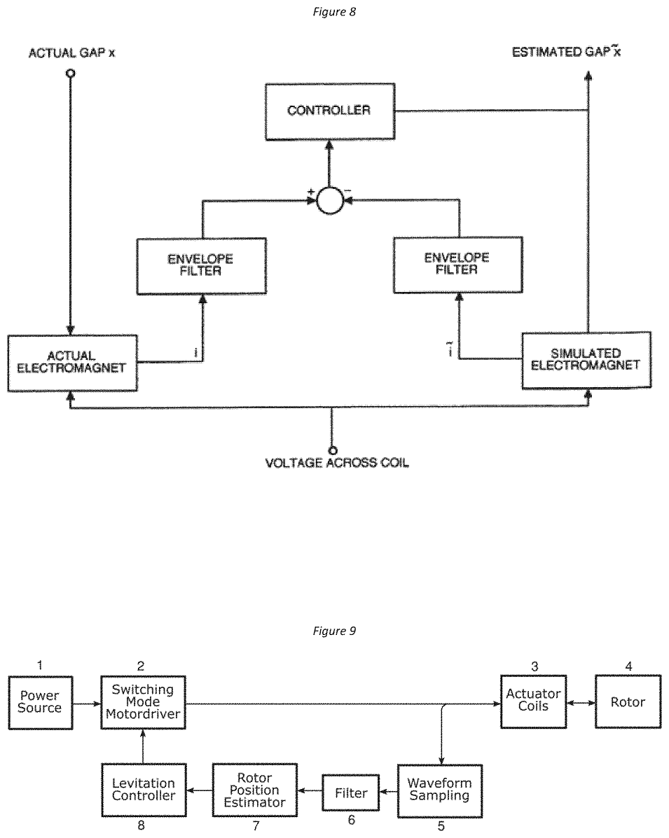

The patent document U.S. Pat. No. 6,302,661B1 describes a method to generate an electronic signal related to rotor position in a linear DOF through a combination of current and voltage provided by the activating coils. The voltage and current waveform (as shown in the patent document U.S. Pat. No. 6,302,661B1) to be measurable in the activating coils are displayed in FIG. 7. The intended method (see FIG. 8; also taken from U.S. Pat. No. 6,302,661B1) to extract the rotor position, in this case the bearing gap, uses an envelope filter on the current measurements to condition the signals from the sensors and uses a control loop with a simulation model of the electromagnet to estimate the bearing gap.

Efforts to replicate the presented rotor position measurement described in U.S. Pat. No. 6,302,661B1 have shown that a rotor position signal is in fact detectable. However, the signal noise and measurement delay have to meet certain requirements to be able to levitate a rotor based on the measurement. Both requirements are more demanding for a smaller and lighter rotor. Therefore, the requirements for levitation of an implantable paediatric VAD system are especially hard to meet. The measurement methods described in U.S. Pat. No. 6,302,661B1 generate too much noise.

The disclosure presents novel methods to reduce the measurement noise at various stages in the measurement chain.

Furthermore, VAD systems, in particular when operated outside a tightly controlled hospital environment, are constantly subjugated to a broad variety of external disturbances which, in turn, are detrimental to the operational safety of the VAD system.

Those external disturbances are mainly of an electromagnetic nature and may thus particularly include electromagnetic interferences, originating from sources such as mobile phones, RFID, CT-scanners, etc. In consequence, such disturbances may interfere with sensors and their related sensor signals which are involved in the various stages of the measurement chain.

The disclosure also presents novel methods to increase the operational system safety of VAD systems.

Every levitation control loop needs a rotor position signal at its input. Commonly, dedicated sensors such as magnetic field sensors (Heart Mate 3, Abbott) or eddy current sensors (INCOR, Berlin Heart GmbH) are used to measure the rotor position. The sensor signal is either evaluated inside the pump with integrated pump electronics or transmitted to the control unit, using additional wires in the driveline. Both options reduce the viability of the VAD system for use in children.

The presented sensor solution does not require dedicated sensors, complex electronic systems inside the pump or the addition of wires to the driveline. In most cases, the motor structure is directly or indirectly used to measure the rotor position.

The presented concepts were developed for a disc-shaped axial flux motor. However, the sensing methods can be used with other types of BLDC motors or even linear actuators e.g. voice coils.

FIG. 9 shows an exemplary block diagram of a VAD with sensorless magnetic levitation. A power source 1 provides electric power to a switching mode motor driver 2, for example an inverter providing a PWM signal to actuator coils 3. The signal provided by the switching mode motor driver 2 corresponds to a voltage across the actuator coils 3 and an electric current within the actuator coils 3 resulting in a magnetic field effectuating a force on the rotor 4. As depicted in FIG. 9, the waveform of the electric current within the actuator coils 3 and/or the waveform of the voltage across the actuator coils 3 may be sampled by a waveform sampling unit 5 and subsequently filtered with a filter 6, for example a low pass filter. From the filtered waveforms, e.g. by evaluation a phase offset between current and voltage, it is then possible to estimate the rotor position with a rotor position estimator 7. Based on the estimated rotor position a levitation controller 8 may calculate appropriate control signals to control the switching mode motor driver 2 such that the actuator coils 3 may be provided with signals suitable to levitate the rotor 4.

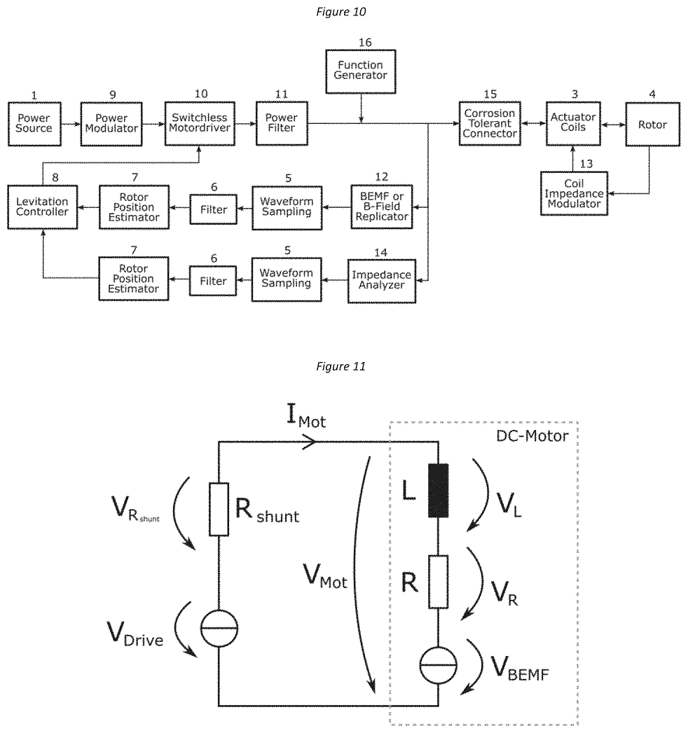

Referring now to FIG. 10 which provides an overview of possible processing blocks of the VAD.

The actuator coils 3, which may be motor coils, are located inside the VAD and are magnetically coupled to the rotor magnets disposed on the rotor 4. The rotor magnets may be arranged as a Halbach array. With the rotation of the rotor 4 a BEMF is induced in the actuator coils 3. The magnitude of the induced BEMF depends on the rotation speed and the distance between rotor magnets and actuator coils 3, that is, on the position of the rotor in z direction which is also denoted as axial direction and which may be an unstable DOF (degree of freedom). It is therefore possible that the position of the rotor is estimated from the BEMF. Since the BEMF cannot be measured directly during motor operation, its, in general, time dependent value needs to be estimated.

The position of the rotor 4 along an unstable DOF modulates the shape of the BEMF and also induces, in addition to the BEMF corresponding to the rotor rotation, a further BEMF into the motor coils 3. The BEMF's from rotor rotation and translation in z direction are modifying the motor phase currents and terminal voltages. Both can be estimated by a BEMF replicator 12, which generates a digital or analog copy of the induced BEMF. The BEMF replicator 12 can further be used to generate an electric or digital signal with the shape, that is, waveform, of the magnetic field within the actuator coils 3. The replicated waveforms are fed via a waveform sampling 5 and a filter 6, as explained in FIG. 9, to a rotor position estimator 7 that estimates the linear and/or rotational rotor position. The method used by the rotor position estimator 7 can include an arithmetic formula that inverses the law of induction, a Kalman filter based on a motor model or a controller controlling a simulation model of the motor. The rotor position is used by the levitation controller 8 to control the motor driver 2 (see FIG. 9) or 10 to adjust forces on the rotor to levitate the rotor within the VAD.

It may also be considered to use the positions of the rotor 4 as an input to a coil impedance modulator 13 which is configured to modulate the impedance of the actuator coils 3. Furthermore, the actuator coils 3 may be connected to a motor driver, for example a switchless motor driver 10 as in FIG. 10, via a corrosion tolerant connector 15 and, optionally via a power filter 11. The motor driver, for example the switchless motor driver 10, may receive electric power from a power source 1 via an optional power modulator 9.

A function generator 16 may imprint a current signal, e.g. a sinusoidal signal, into the motor coil. This signal imprinted by the function generator 16 is picked up by the BEMF-Replicator 12 only if the BEMF parameters, for example the motor coil impedance estimate, are not perfectly chosen. Therefore, if the output of the BEMF-Replicator 12 correlates with the imprinted signal, then the BEMF-parameters have to be adjusted. The phase and amplitude of the correlation indicate, in which direction the parameters have to be adjusted. The imprinted signal may be a high frequency signal, for example with a frequency greater than the rotational frequency of the rotor. The imprinted signal may as well be modulated. The modulated signal may be used as a test signal.

BEMF-based rotor position sensing doesn't generate a non-zero signal when the rotor is at standstill. Therefore, the rotor has to be spun before the levitation control can detach the rotor and levitate the rotor.

However, the BEMF parameter estimation using the externally imprinted signal imprinted by the function generator 16 is operational even when no BEMF is present, i.e. the rotor does not rotate.

When the distance between rotor-magnets and motor coils, that are disposed within the motor stator, is increased, then less magnetic flux reaches the motor coils. This affects the characteristic of the motor setup. Characteristic motor parameters are e.g. the no-load-speed and stall torque. A weaker field or greater rotor-stator-distance will increase the no-load-speed and decrease the stall torque. During operation, neither of these parameters can be measured directly. However, both are defining the current, voltage and speed behaviour of the motor. A measurement of either of these values can be used to calculate an estimation of the BEMF.

The BEMF is related to the magnetic field through the motor coils via the law of induction:

$${V}_{BEMF} = - N \frac{d Φ}{dt} = - N \frac{d}{d t} ( {\int }_{A} B d A ) \qquad ( 1 )$$

with N—number of windings, ϕ—magnetic flux through coils.

The field outside of a linear Halbach array rotor can be estimated to:

B(x,z)=B₀e^{ikx}e^{-kz} (2)

with B_0 as magnetic field strength at the surface of the magnets, k as magnet wave number and z as the distance from magnet surface, wherein in a Halbach array magnets are specifically arranged along a line and wherein x denotes a displacement of the rotor into the direction of the line of the Halbach array.

When arranging the magnets of a Halbach array as a ring, by inserting (2) in (1) and in the case of a rotating rotor with a rotor speed ω, the BEMF is substantially proportional to field strength and electric rotor speed ω:

$${V}_{BEMF} ( x , z = const ) = - {Nc}_{geo} \frac{d}{d t} ( {B}_{0} {e}^{i ω t} {e}^{- kz} ) = - {Nc}_{geo} {B}_{0} {e}^{- kz} \frac{d}{dt} ( {e}^{i ω t} ) \qquad ( 3 )$$

with c_geo as a coil geometry constant and t as time.

The actually measurable BEMF is:

$$( 4 ) = re ( Equation 3 )$$

$$re [ {V}_{BEMF} ( t , z = const ) ] = - {Nc}_{geo} {B}_{0} {e}^{- kz} \frac{d}{d t} cos ( ω t ) = {Nc}_{geo} {B}_{0} {e}^{- k z} ωsin ( ω t )$$

where re(⋅) denotes the real part of a complex number.

With known speed and a known, for example estimated, BEMF value, the rotorposition in z direction can be calculated. However, the BEMF cannot be measured directly while the motor is in operation under field oriented control. The only measurable motor voltage is the sum of BEMF, voltage across coil resistance and voltage across coil inductance (V_{Mot}, V_{BEMF}, V_{R}, V_{L} in FIG. 11). Using a measurement of the motor current I_{Mot}, the voltages inside the motor, V_{L} and V_{R} can be estimated by calculation. After having measured the voltage between the terminals of the motor coil V_{Mot}, an estimation of V_{BEMF} can be calculated. FIG. 11 shows an equivalent circuit of a motor coil including a series connection of the coil inductance L with the corresponding voltage V_{L}, the coil resistance R with the corresponding voltage V_{R} and the BEMF voltage source V_{BEMF}, and in parallel to the motor coil a shunt resistor Rₛₕᵤₙₜ with the corresponding voltage V_{Rshunt} and a drive voltage source V_{Drive}. The motor coil terminal voltage V_{Mot} is the sum of V_{L}, V_{R} and V_{BEMF} and the current through the motor coil is denoted as I_{Mot}.

The motor speed can be estimated e.g. with a frequency analysis of the BEMF. With these measurements, one obtains

$${e}^{- k z} sin ( ω t ) = {c}_{zest} \frac{{V}_{Mot} - {RI}_{Mot} - L \frac{{dI}_{Mot}}{dt}}{ω} \qquad ( 5 )$$

with c_zest—z position estimation constant.

Every phase of a three phase BLDC motor can be modelled according to FIG. 11. It can be sufficient to measure only one phase to gain a z-position measurement. However, by measuring all three phases a more accurate measurement can be performed. For example, equation (5) can be evaluated for each of the motor phases and combined to a space phasor with an angle and an amplitude from which the z-position of the impeller may be estimated. Equations (3), (4) and (5) are approximations for quasistatic changes of the axial rotor position z. For a more dynamic estimation of the z position, the errors introduced by the approximations can be compensated. Known methods for compensation include numerical integration of the BEMF to generate coil flux estimations, sliding mode observers or PLL-based observers which directly estimate the rotor position.

An accurate measurement of the DC-equivalent motor-current of a BLDC motor can be taken by measuring the current in the supply of the commutator circuit (see, for example, FIGS. 12a and 12b).

These methods are accurate enough to commutate the motor using the estimated BEMF and also accurate enough to get an averaged z-position measurement. However, if fast and accurate measurements of the BEMF are necessary, e.g. for a position control loop in a levitation device, then improvements may be necessary to increase the signal-to-noise ratio.

The presented improvements for measuring the BEMF are reducing the systematic noise of the current measurement, voltage measurement, the subsequent filtering and the position estimation. Especially the high frequency noise reduces the accuracy of the inductance voltage estimation. This is due to the high-pass characteristic of the differentiation operation in the inductance voltage estimation.

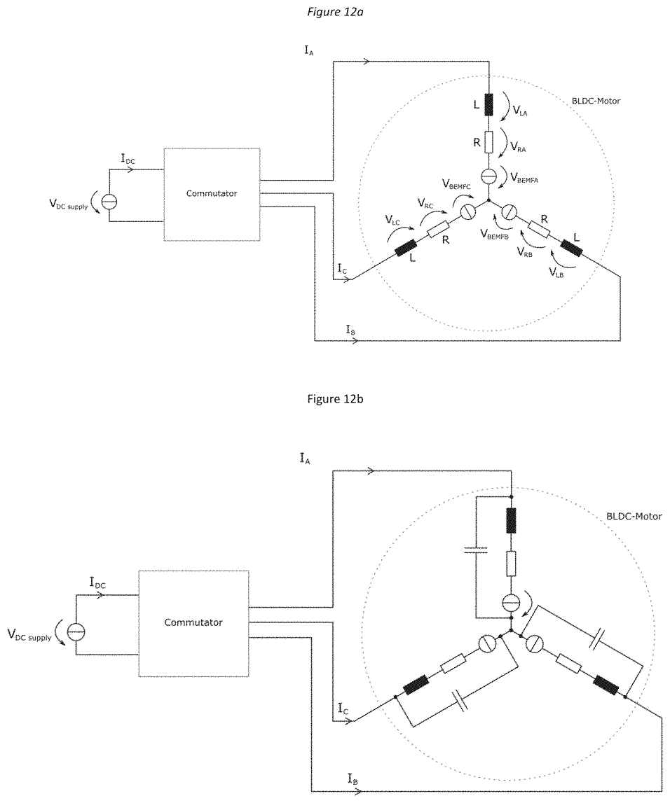

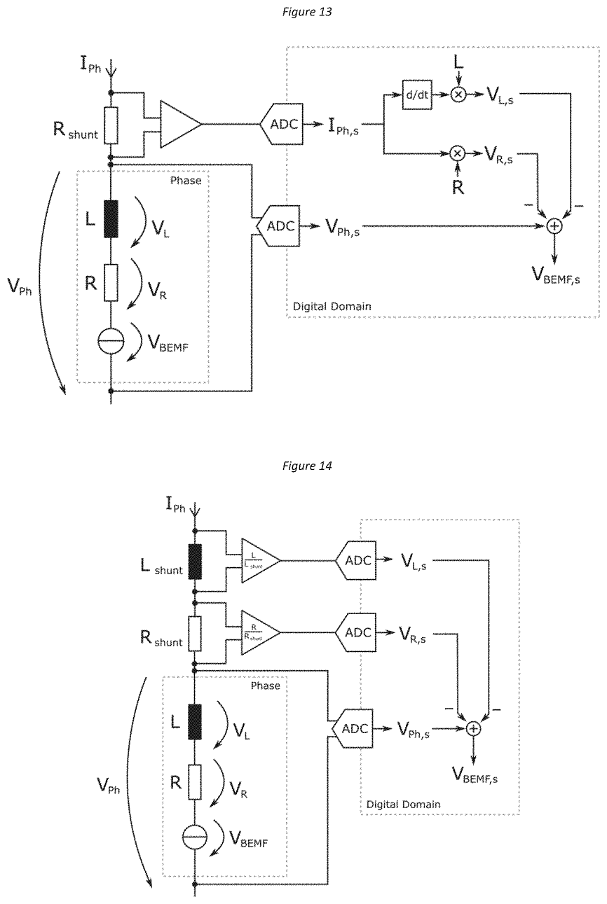

A circuit for calculating the BEMF is shown in FIG. 13. The waveform of the voltage across a motor coil V_{Mot}, here denoted as phase voltage V_{Ph}, is sampled with an analog digital converter and hence converted into a digital signal V_{Ph,s}. Similarly, the voltage across a shunt resistor Rₛₕᵤₙₜ connected in series with the motor coil, which is proportional to the electric current I_{Ph} through the shunt resistor and the motor coil, is sampled with an analog digital converter and hence converted into a digital signal I_{Ph,s}. Before sampling the voltage across the shunt resistor, the voltage may be amplified, wherein the amplifier may have a high input impedance. I_{Ph,s} is processed in a first and a second processing path. In the first path, I_{Ph,s} is derived with respect to time and multiplied with the (estimated) value of the coil inductance to calculate an estimate V_{L,s} of the voltage V_{L} across the coil inductance L. In the second path, I_{Ph,s} is multiplied with the value R to obtain an estimate V_{R,s} of the voltage across the coil resistance V_{R}. Eventually, an estimate V_{BEMF} of the BEMF in the motor coil is obtained with V_{BEMF}=V_{Ph,s}−V_{R,s}−V_{L,s}.

The circuit for calculating the BEMF as shown in FIG. 13 has limitations. Embodiments to overcome limitations of the circuit of FIG. 13 will be explained below.

The circuit in FIG. 14 is similar to the circuit in FIG. 13. However, in FIG. 14 an inductive shunt Lₛₕᵤₙₜ is connected in series to the resistive shunt Rₛₕᵤₙₜ. In contrast to FIG. 13, the voltages across Rₛₕᵤₙₜ and Lₛₕᵤₙₜ are amplified, wherein the respective amplifiers may have a high input impedance, and subsequently converted in digital signals V_{R,s} and V_{L,s}, respectively. The motor current and its transients also flow through the resistive and inductive shunt. The voltage across the resistive shunt is a scaled image of voltage across the motor coil resistance. The voltage across the inductive shunt is a scaled image of voltage across the motor coil inductance. Hence, an analog voltage sample of V_{R} can be generated by amplifying (rescaling) the voltage across the resistive shunt. An analog voltage sample of V_{L} can be generated by amplifying (rescaling) the voltage over the inductive shunt. Therefore, with appropriate amplification, first path and second path of FIG. 13 may be simplified in FIG. 14. That is, V_{R,s} and V_{L,s} are available directly as the signals provided by the respective analog-digital converters sampling the amplified (rescaled) voltages across Rₛₕᵤₙₜ and Lₛₕᵤₙₜ, respectively. As in FIG. 13, the BEMF estimate may be calculated with V_{BEMF}=V_{Ph,s}−V_{R,s}−V_{L,s}. Appropriate amplification factors in this example may be L/Lₛₕᵤₙₜ for the inductive shunt voltage and R/Rₛₕᵤₙₜ for the resistive shunt voltage.

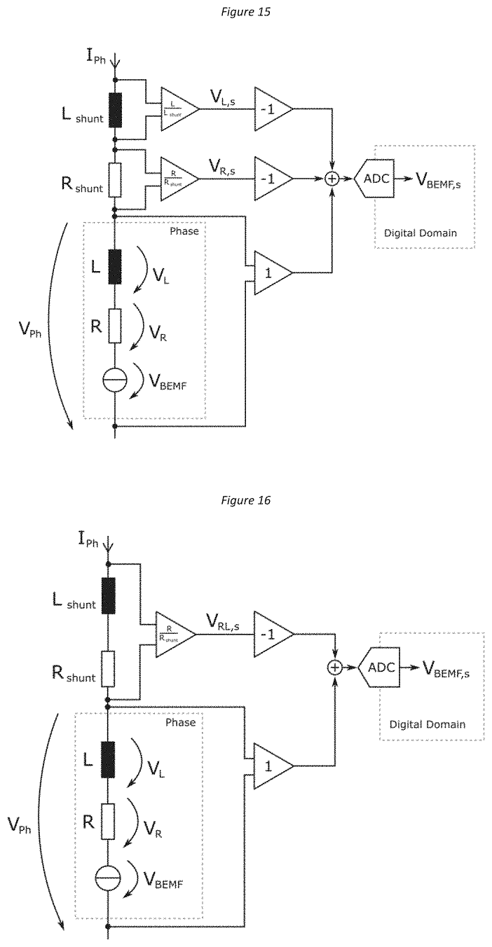

Instead of calculating the BEMF voltage V_{BEMF} in the digital domain as in FIG. 13 or 14, the calculation of V_{BEMF} can also be realized in the analog domain as shown in FIG. 15. In FIG. 15 the mathematical operation V_{BEMF}=V_{Ph,s}−V_{R,s}−V_{L,s} is performed in the analog domain, for example using an analog adder with an operational amplifier, and transformed into the digital domain afterwards with an analog digital converter.

The digital implementation of the BEMF estimation (FIGS. 13 and 14), that is the generation of a BEMF replica, may suffer from convolution of above Nyquist frequency disturbances into the signal frequency range (aliasing), due to the limited sampling frequency of the analog digital conversion. The subsequent computation amplifies these convoluted disturbances. The analog generation of a BEMF replica (FIG. 15) does not suffer from this problem.

FIG. 16 presents a circuit with a further simplification of the circuit in FIG. 15: If the ratio between resistive shunt and inductive shunt is equal to the ratio between motor resistance and motor inductance, then only a single combined resistive-inductive shunt can be used. To precisely match the impedance, resistance and also stray capacitance, a physical copy of the motor coil can be used. The copy can be exact or scaled in dimension. By using only one complex impedance shunt instead of a resistive shunt and an inductive shunt, fewer amplification parameters have to be set correctly to replicate the BEMF. In the example shown in FIG. 16, the amplification factor may be R/Rₛₕᵤₙₜ.

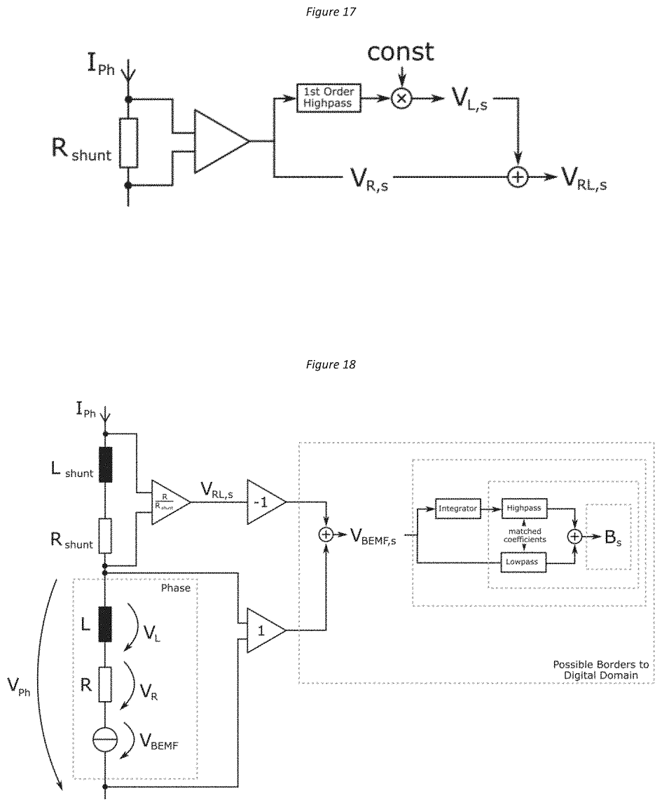

FIG. 17 shows a further simplification in comparison to FIGS. 15 and 16. Instead of capturing the voltage across the resistive shunt and the inductive shunt or a combination thereof, it may be sufficient to capture the voltage across the resistive shunt alone. The inductive part may then be emulated with a high pass filter, for example a 1^{st} order high pass filter, which may have a similar transfer function as the transfer function of an inductive shunt which is equal to analog differentiation. Differences in scaling may be equalised by an amplification of the output signal of the high pass such that the amplified and high pass filtered signal is an estimate V_{L,s} of the voltage across the motor coil inductance V_{L}. The voltage as captured across the resistive shunt may serve as an estimate V_{R,s} of the motor coil resistance voltage V_{R}. Hence, an estimate of the voltage across motor coil resistance and inductance may be obtained with V_{RL,s}=V_{R,s}+V_{L,s}. The BEMF voltage may then be estimated from V_{BEMF}=V_{Ph,s}−V_{RL,s}. Omitting the inductive shunt is advantageous since the inductive shunt requires physical space and creates additional losses.

The values of L, R_shunt and L_shunt have to be known to choose an optimal value for the amplification factor, denoted as ‘const’ in FIG. 17, before adding up all voltage components to calculate the BEMF.

Clearly, the operations described above and shown in the FIGS. 15 to 17 may also be implemented in the digital domain with circuits similar to FIGS. 13 and 14 or parts of those circuits as appropriate.

If the amplification factor is not optimal, then the phase current and phase current derivative are cross-talking into the BEMF signal. The optimal amplification factor can be determined during a factory calibration. However, temperature dependencies or aging may change the values of R, L, Rₛₕᵤₙₜ or Lₛₕᵤₙₜ over time.

In a VAD system it is not possible to stop the pump just for a calibration. Therefore, a calibration or re-calibration or measurement of the components R, L, Rₛₕᵤₙₜ and Lₛₕᵤₙₜ or the optimal amplification factor has to be performed while the blood pump is in operation.

Now referring to FIG. 45. In the context of the presented solution an additional test signal is added onto the phase current I_{Ph}. The added signal needs to be a signal that can be separated from the normal motor operation current. At the same time, this signal should not interfere with the motor operation or levitation control. Options for this signal include a repetitive waveform with a specific frequency, preferably significantly higher than the necessary levitation control frequency or, a sufficiently low amplitude pseudo-random signal below the levitation control frequency.

The test signal can be added, using the already available hardware of the motor driver, that is, by requiring no further hardware modifications.

If the BEMF amplification factors are not optimal, then the phase current cross-talks into the BEMF estimation. The added test signal can be isolated from the BEMF estimation e. g. with frequency filtering, e.g. lock-in detection or, with correlation.

Based on the cross-talk measurement, a negative feedback controller adjusts the amplification constants, so that the cross-talk is reduced. The two necessary amplification factors can be distinguished by the phase of the cross-talk. The cross-talk signal related to the amplification factor related to R/Rₛₕᵤₙₜ has no phase shift with respect to the phase current and the cross-talk signal related to the amplification factor related to L/Lₛₕᵤₙₜ has a phase shift of 90 degrees for an ideal inductor and less for a real one.

The presented method for amplification factor control does not need a physical inductive shunt. It can also be used to adjust the amplification factors in BEMF estimation methods as shown in FIG. 13 (amplification factors L and R in digital domain).

The estimations according to equation (1) to (5) are only valid for slow movements in the z-direction. A magnetically destabilized z-axis can reach speeds where the change in magnetic field also induces a significant voltage in the motor windings.

This leads to wrong rotor angle and z-distance estimations when conventional BEMF vectors are evaluated.

To detect fast movements in the z-direction, it is advantageous to know the actual magnetic field strength inside the coil windings of a motor phase.

Due to the relation between BEMF and magnetic flux:

$${V}_{BEMF} = - N \frac{d Φ}{dt} \qquad ( 6 )$$

the flux can be calculated from the BEMF:

$$Φ = - {\int }_{t} \frac{{V}_{BEMF}}{N} dt = B A \qquad ( 7 )$$

with ϕ—flux, N—windings, A—coil area, B—B-field (magnetic flux density) of the coil.

The integration creates an offset due to the unknown integration constant. Therefore, only the high pass characteristic of the flux can be determined with high accuracy. However, the low pass characteristic of the z-axis movement can be extracted directly from the BEMF measurements.

In order to obtain the high pass characteristic of the flux, the flux as calculated with equation (7) may be filtered with a high pass. Furthermore, in order to obtain a low pass characteristic of the position signal the estimated BEMF may be low pass filtered with a low pass and additively combined with the high pass characteristic of the flux. With this combination a fast and accurate rotor position estimate may be obtained. As an option, the low pass and the high pass may be designed to match each other, that is, to form a matched pair. As an example, the low pass and the high pass may be complementary filters whose transfer functions add up to a constant value, also known as a complementary filter pair. The integration and filtering with filters matched to each other can be accomplished both in the analog and digital domain.

FIG. 18 shows the flow chart and possible transitions from analog to digital domain. The circuit in FIG. 18 shows, as an example, the estimation of the BEMF voltage V_{BEMF,s} according to the method of FIG. 16. The BEMF voltage may be determined by another method as well, for example any of the methods described above or depicted in the FIGS. 13 to 17. The processing of the estimated BEMF voltage, as exemplarily shown in FIG. 18, may be accomplished with a first signal path including an integrator and a high pass filter and with a second path including a low pass filter. The filter parameters of the high pass filter and the low pass filter may be matched such that the high pass filter and the low pass filter are a matched pair of filters. The output signals of the high pass filter and the low pass filter are added to receive an estimate Bₛ of the B-field of the coil.

The described filtering method combines the advantage of the BEMF-based sensor that is an integrator and integrator-drift free output signal, with the advantage of the B-Field calculation that is a good low noise distance signal even for fast axial movements.

The rotor position can be directly calculated from the magnetic field strength using formula (8):

B(x,z)=B₀e^{iωt}e^{-kz} (8)

Now further referring to FIG. 45. In order to be able to accurately replicate the BEMF the motor parameters coil inductance L and winding resistance R need to be known precisely. They may be determined during factory calibration. However, they may change their values afterwards, for example depending on temperature or due to aging. It may hence be necessary to re-determine and/or to track these parameters before an implantation and/or during operation of the blood pump.

The subsequently described method determines an estimation error of the parameters for a motor in operation or at stand still and adapts the motor parameters using a control loop (servo loop). For this, the BEMF and the magnetic flux are continuously determined according to equation (1) and equation (7), respectively. In addition, a test signal may be injected (see FIG. 45). The test signal may include frequency components sufficiently high with respect to rotor moment of inertia to prevent or at least to strongly attenuate a rotor response to the test signal due to magnetic field induced by the electric current corresponding to the test signal. The test signal may be generated, for example, by the function generator 16 of FIG. 10. In FIG. 45, the test signal is fed into the motor coil via a resistive shunt and into a test signal processing block comprising a detector or a correlator or a filter. The BEMF computation which may implement a method or a device for BEMF estimation as described above is fed with the values of the voltages across the motor coil and the resistive shunt. The test signal processing block is configured to estimate the accuracy of the estimate of the motor parameters, that is, the impedance of the motor coil, from the amplitude of the test signal within the estimated BEMF by, for example, way of correlation. The rationale of this method is that a poor estimate of the motor parameters coincides with a high correlation value and a good estimate coincides with a low correlation value between estimated BEMF and the test signal. The test signal processing block provides information to a controller on how to adjust the motor parameter estimate. Therefore, the accuracy of the estimate of the motor parameters is continuously adjusted resulting in a continuous adaption of the BEMF estimation with the objective to improve the accuracy of the BEMF estimate which may vary with operation conditions of the blood pump.

Since the rotor may not follow the test signal, the induced BEMF (B-BEMF in FIG. 45) does not contain spectral components in the frequency range of the test signal. So, if the BEMF replica is estimated with correct motor parameters, for example the impedance of the motor coils, then the estimated BEMF replica will not contain spectral components of the test signal as well or contain a known reduced amount of the spectral components. Though, in this case, V_{L}, V_{R}, and V_{Ph} would include components of the test signal, these components would be cancelled to zero when calculating the BEMF replica. However, with inaccurate motor parameters these components of the test signal will be not perfectly cancelled such that a component of the test signal will remain within the estimated BEMF replica. The impact of the test signal on V_{L} and V_{R} (FIG. 45) differs with respect to the inductive phase offset. This phase offset does still exist in the estimated BEMF replica. A corresponding detector, for example a correlator or filter, may detect from the BEMF replica an error signal indicative for the parameter L and an error signal indicative for the parameter R. Each of the error signals is passed to a controller configured to adjust the respective motor parameter in order to reduce the respective error signal. The controller may be implemented, for example, as an I-controller.

The test signal is required to prevent the rotor from following the test current and to allow a good detectability of the test signal from the BEMF replica and a good differentiation from external disturbances. Possible test signals are, for example, sinusoidal signals above the operating frequency of the motor. They may, for a better differentiation of the BEMF replica from external disturbances, be amplitude modulated, phase modulated or frequency modulated. A combination of a plurality of frequencies is conceivable as well. Alternatively it is possible to use random and/or pseudo random signal for modulation (e.g. a Gold code, maximum length sequence), as long as it includes sufficiently high frequency components. Such a signal may be better filtered from the BEMF replica with a correlator or synchronous detector.

If the frequency range of the test signal overlaps with the frequency range of the rotor movement, then it is possible to estimate and compensate the BEMF replica due to the test signal using a mechanical model. Said mechanical model comprises model parameters, for example the rotor mass, moment of inertia, spring constants and/or friction coefficients. These parameters may be stored in the control unit and may be validated or corrected during start-up or during another dynamic process.

The advantageous effects of this method are:
Calibration during operation possible
Calibration possible without rotor movement
Continuous compensation of drift (shunt, amplifier, current sensor, analog-digital converter, etc.)
Temperature compensation of, for example, coil resistance, drive line resistance, plug resistance, shunt resistance, shunt amplifier, analog-digital converter
No additional hardware required;
Detection of wear/corrosion, for example of a driveline, a plug or a coil insulation, based on parameter drift

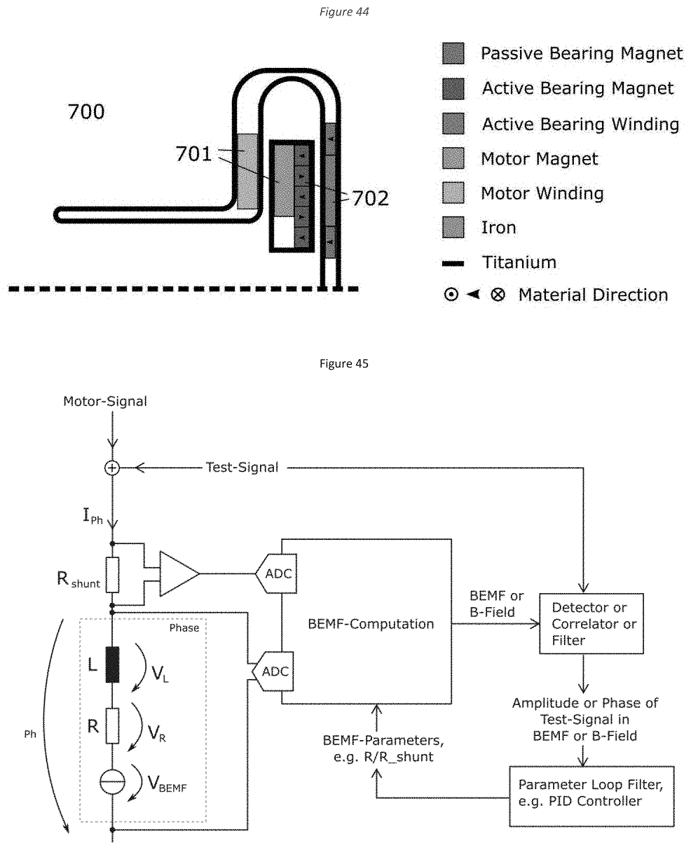

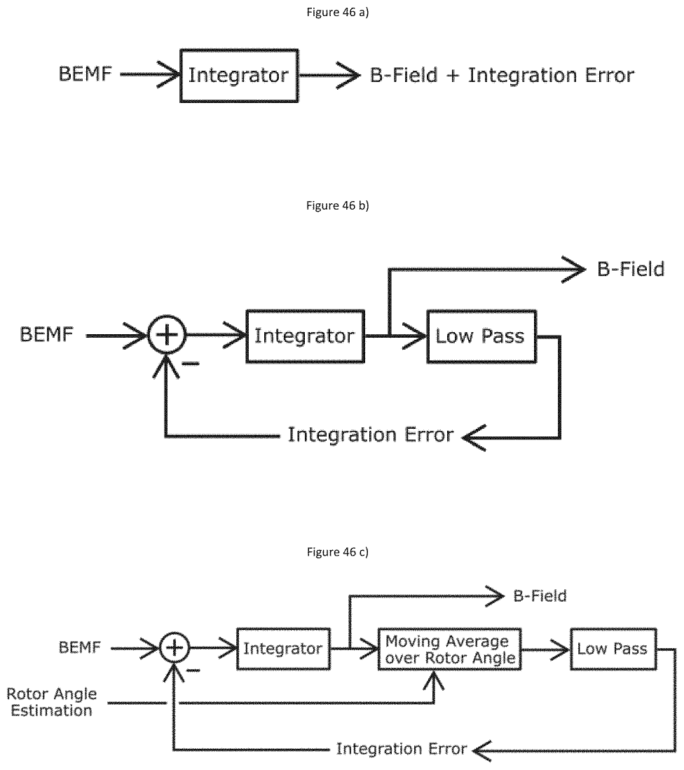

When integrating the BEMF to obtain the B-Field, an integration error may be generated (FIG. 46 a)), for example leading to an offset which may increase over time (drift).

This error can be minimised, using the above-mentioned matched pair of a high-pass and a low-pass filter. However, it is possible to take advantage of the fact that the B-Field over a longer time period oscillates around zero. Therefore, any integration error can be filtered from the output using a high-pass filter.

This method on its own is numerically unstable. The high pass filter behind the integrator can only operate as long as the integration constant is within certain limits. However, without further measures the integration constant can increase indefinitely. Instead, a low-pass filter is usually used, to determine the integration error which is then used in a self-regulating servo-loop (FIG. 46b)). The servo loop prevents the accumulator inside the integrator from increasing to values much greater than the input samples, which would lead to large rounding and integration errors. Hence, in FIG. 46b, the integrator output is fed into a low pass and the low pass output is fed back to integrator input where it is subtracted from the BEMF which is also an input signal to the integrator. The integrator output is an estimate of the B-Field.

The disadvantage of a servo-loop according to FIG. 46b) is that the low-pass filter has to have a sufficiently high cut-off frequency, to be able to follow the integration error. This, however, may limit a band width of the B-Field estimate at the integrator output, that is, the estimated B-field at the integrator output may not follow the actual B-field in the motor coil. To enhance the band width of the estimated B-field, that is, to improve the dynamic behaviour of the servo-loop, another characteristic of the BEMF can be used to estimate the integration error faster.

So, as a further specific characteristic of the BEMF signal it may be observed that the integral of the BEMF is zero not only when calculated over time, but also when calculated over the rotation angle for an entire electrical and/or mechanical revolution. A moving average filter can be used to determine the average integral BEMF over an electrical and/or mechanical revolution. The output of the moving average filter is an approximation of the integration error with low latency. The subsequent low-pass filter can have a much lower cut-off frequency than the low-pass filter in FIG. 46b), which leads to a better dynamic response of the B-Field in FIG. 46c).

The moving average filter in certain, computationally efficient, embodiments can also suffer from instabilities, when floating point arithmetic is used in combination with an accumulator. Numerical stable implementation of the moving average filter either uses a fixed point arithmetic or resets the accumulator regularly or even in each time step.

Referring again to FIG. 10. An additional or alternative measurement method to BEMF replication may be used to determine the rotor position. The following methods also enable detachment at standstill, wherein detachment refers to moving the rotor, for example, from an off-centred position to a centred position within the blood pump. With respect to FIG. 10, the rotor position estimation 7 based on signals provided by an impedance analyser 14, waveform sampling 5 and filter 6 are now described. The rotor position, as estimated using subsequently described methods may also be used as an input signal to the levitation controller 8. The impedance analyser 14 is fed with a voltage and/or an electric current signal corresponding to the respective signals provided to the actuator coils 3. These signals may include components generated by the function generator 16, for example a high frequency signal and/or a test signal including a modulated high frequency signal.

In a first method, the high frequency impedance of the actuator coils 3, which may be motor coils, is influenced by the rotor position. The actuator coils 3 have an inductance but also a certain amount of capacitance between the turns. This generates an RLC circuit forming a resonant circuit having a resonance frequency. The resonance frequency is usually located at several MHz and well above common PWM frequencies. The resonance frequency and quality factor (corresponding to a 3 dB bandwidth of the resonance) can be influenced by the rotor position due to magnetic coupling. Any high frequency magnetic field generated by the actuator coils 3 can generate an eddy current inside conductive parts of the rotor 4. The generated eddy current creates its own magnetic field that opposes the magnetic field generated by the actuator coils 3, modifying their impedance. This mechanism is commonly described as eddy current sensor and is state of the art, when used with a dedicated sensing coil. In certain aspects, however, the actuator coils 3 are used as eddy current sensors.

Coreless and/or yokeless motor windings are especially suitable for this application, because eddy currents will be introduced into the core and yoke. This effect may degrade the signal-to-noise ratio.

Eddy current sensors detect the presence of a conductive target in the field of a high frequency coil. Normally, motor coils are not suitable for eddy current measurements, because they would primarily detect the presence of the iron core. In a further aspect, it has been found that ironless (stator-core- and yoke-less) motors do not have this problem, but still have enough efficiency to drive the pump. In this case, the windings of ironless motors can be used to take eddy current readings. The target could be a copper plate in the rotor, the rotor titanium housing or conductive magnet material.

The negative effect of the iron core can be reduced by laminating the core from several thin isolated metal sheets or by utilizing a sintered ferrite core.

In operation, a high frequency current is imprinted on the motor coil current. The motor driver and eddy current sensing circuit can be isolated from each other using passive filters. A large gap between eddy current frequency and motor operation frequency or PWM frequency benefits the filter design.

Active filters, e.g. lock-in amplifier, could also be used for the extraction of the eddy current signal. One of the biggest noise sources are the harmonic components of the PWM signal. Motor drivers with no or reduced switching noise are especially suitable for simultaneous eddy current measurements. Such drivers are explained further below.

BEMF sensing cannot be used at very low speeds. This means, that the axial rotor position cannot be measured with BEMF below a certain speed. A maglev pump that uses only BEMF to measure rotor angle and axial position would need to spin the rotor before lift-off. Sufficiently good backup bearings, that support the rotor when the magnetic bearing does not support the rotor, are necessary to allow this type of operation.

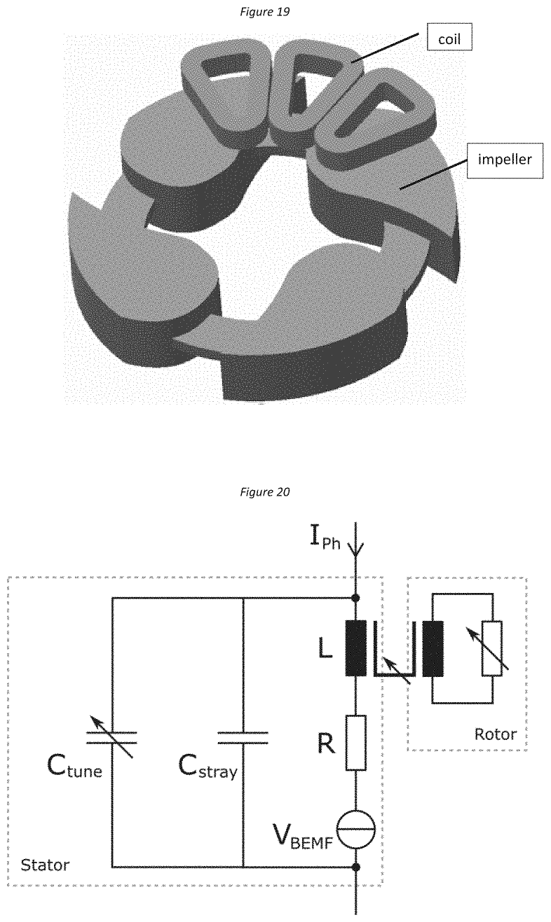

Advantageously, eddy current sensors do not rely on a rotation of the rotor and are generating a signal even at 0 rpm. The rotational rotor position could be measured by comparing multiple eddy current sensors in multiple motor phases (FIG. 19). The rotation detection could even be implemented in a shrouded rotor. Dedicated copper targets could be placed on top of some of the magnets, making them better targets. An alternative or addition to this is to reduce the conductivity of some magnets by segmenting and isolating the magnets. This method is used excessively in high performance BLDC motors to reduce eddy current losses.

To improve the signal-to-noise ratio, the BLDC commutation could be operated in six step mode, where one of the phases carries no motor current. In each step two phases are energised by the motor driver and the third phase is floating. This phase, that is, the motor coil of this phase, is then used for the eddy current measurement by imprinting a high frequency current. Only the measurement current is flowing in the third phase. This yields the advantage that the eddy sensor measurement current does not require to be filtered from the motor current. Many eddy current sensing circuits that normally require a dedicated sensor coil can now be used with motor coils while, at the same time, operating the motor. Examples for these sensing circuits are balanced impedance bridges or resonant circuits.

FIG. 12a shows the electrical equivalent circuit of a brushless direct current (BLDC) motor including a DC power supply and a commutator and three substantially equal phases A, B and C wherein each phase comprises a motor coil, as depicted in FIG. 11 and described above, having an inductance L, a resistance R and a Back-EMF (BEMF) voltage source. Each phase A, B and C may include a phase current I_{A}, I_{B} and I_{C}, respectively. One terminal of each motor coil is connected to the commutator and respective other terminals of the motor coils are electrically connect with each other (Y connection). The commutator is provided with electrical energy from a DC voltage supply with a voltage VDC and an electric current IDC.

Similar to FIG. 12a, FIG. 12b shows the electrical equivalent circuit of a brushless direct current motor with a parasitic capacitance parallel to the series connection of a resistor, an inductance and a BEMF voltage source, that is the motor coil, in each motor phase (phase), wherein an electric current I_{A} is in phase A, an electric current I_{B} is in phase B and an electric current I_{C} is in phase C. Resistor, inductance, BEMF voltage source and parasitic capacitance may be considered as an equivalent circuit for a motor coil of a phase wherein resistor, inductance and capacitance form a resonant circuit having a resonance frequency. In addition to the parasitic capacitance, an additional capacitor in parallel to the parasitic capacitance of each phase may increase the capacitance existing in each of the phases thereby lowering the resonance frequency of the respective resonant circuit. Lowering the resonance frequency may have the advantage that the resonance frequency moves to a frequency range where a technical detection and processing of the resonance can be performed more efficiently.

Furthermore, by choosing different additional capacitors in parallel to the parasitic capacitances of the different phases, the resonance frequency of the resonant circuit in one phase may be different from the resonance frequency of the resonant circuit in another phase such that a specific resonance frequency may be assigned to a specific motor phase. Since each of the motor phases may be realised with a motor coil and the spatial position of each motor coil is known from the motor design, each of the resonances of the resonant circuits corresponds to a spatial location. Therefore, by observing the resonances it may be possible to detect translational and/or rotational changes of the rotor position since the rotor positions may have an influence on the inductance and/or the parasitic capacitance of a motor coil forming the resonant circuit. Due to eddy currents, the rotor position may also have an effect on the losses and hence an effective resistance of the resonant circuit.

Each of said resonant circuits has an impedance, which is in general a complex number and may be interpreted as an impedance of an eddy current sensor formed by a motor coil with a capacitor connected in parallel. In FIG. 12b two out of three motor phases are connected in series, for example phases A and B, A and C or B and C. Due to the series connection, the impedances of the respective resonant circuits add up.

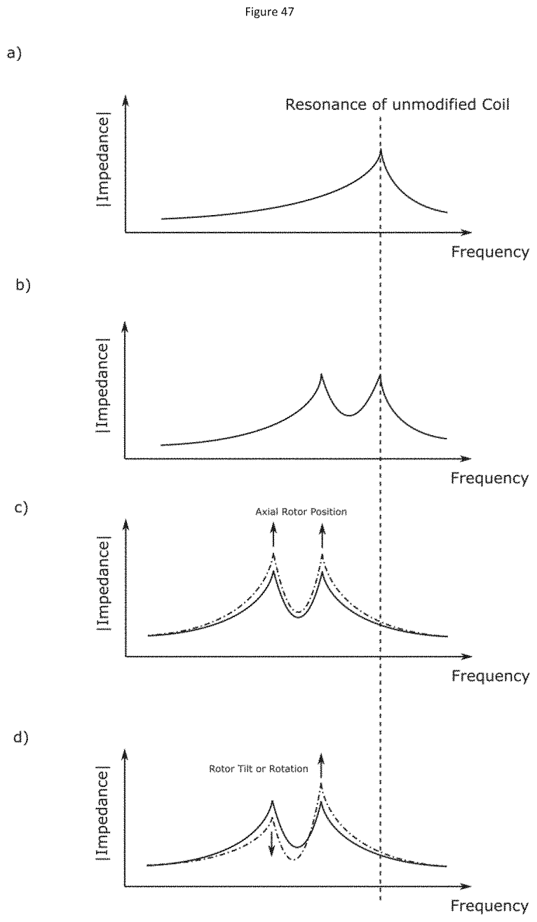

Furthermore, the impedance of said resonant circuit assumes a local maximum at the resonance frequency. FIG. 47a shows a diagram depicting a frequency versus impedance curve of a series connection of the resonant circuits of, for example, phase A and phase B without the additional parallel capacitor in these phases. Since resistor, inductance and parasitic capacitance have substantially the same values due to the motor design both resonant circuits have the same resonance frequency and hence the maximum impedance at the same frequency. Thus, there is only one local maximum in the frequency versus impedance curve.

FIG. 47b shows a similar curve as in FIG. 47a for the case when there is the additional capacitor in phase A in parallel to the parasitic capacitance of phase A. The curve comprises two local maxima: one maximum is at the same frequency as in FIG. 47a, that is at the resonance frequency without the additional capacitor (resonance of unmodified coil) as in phase B and the resonance related to phase A with the additional capacitor in parallel to the parasitic capacitance. Due to the greater total capacitance in phase A, the resonance frequency of the resonant circuit in phase A is less than the resonance frequency of the resonant circuit in phase B.

FIG. 47c shows a curve similar as in FIG. 47b for the case when there are additional capacitors in phase A and phase B and wherein the capacitance of the additional parallel capacitor in phase A is different from the capacitance of the additional parallel capacitor in phase B. There are again two local maxima of the impedance but both maxima are disposed at resonance frequencies that are less than the resonance frequency of a phase without an additional capacitor (unmodified coil). If the rotor is moved then, in general, the inductances and the capacitances of the resonant circuits may change. In particular in axial flux rotors this effect may be seen. As a consequence, the resonance frequency of one or more of the resonant circuits may move to another frequency, the phase angle of the complex impedance may change for a specific frequency and the value of the local maximum at the resonance frequency when depicted in a diagram frequency versus impedance may change, for example become greater or less.

The particular change of the impedance following a movement of the rotor may depend on the type of the movement itself and it may depend on the design of the rotor and/or the stator. For example, in a rotationally symmetric design of an axial flux rotor, for example a rotationally symmetric design of an eddy current target, all impedances may change in the same way when the rotor is axially moved. This effect is depicted in FIG. 47c with the dashed-dotted line where the values of the resonance peaks change in the same manner.

If the rotor is tilted, the impedances of spatially opposing resonant circuits may change in opposite directions. In addition, in case of a rotationally non-symmetric rotor, as for example depicted in FIG. 19 or a non-symmetric application of copper on the rotor, it is possible to detect the angular position of the rotor by evaluating the impedances of the resonant circuits. FIG. 47d shows, similar to the curve depicted in FIG. 47c, but for the latter two cases, a dashed-dotted curve which is an example for a change of the impedance when the rotor is tilted or a rotationally non-symmetric rotor is rotated. In such cases the value of the resonance peaks may change differently, for example one of the resonance peaks may have an increasing value and one of the resonance peaks may have a decreasing value.

Instead of isolating the eddy current sensor signal from the PWM harmonics, it is also possible to use one of the PWM harmonic components to excite the motor coil resonance. Here, a high frequency PWM with short switching times can actually be advantageous. High frequency PWM motor drivers are also optimal for ironless BLDC motors. The low inductance of ironless motors can make additional inductances in the motor driver necessary. These inductances are smaller or, can be omitted completely, in high frequency motor drivers.

The resonance signal could be extracted from the motor signal using lock-in filters which are clocked, directly or indirectly, by the same clock source that also clocks the PWM cycle.

Highly integrated motor driver integrated circuits achieve high switching speeds in small packages, enabling small control units.

The resonance of eddy current sensors can be characterized by the resonant frequency and the resonant quality factor. Most often, the frequency is evaluated. However, in some applications the quality of the resonance can be more sensitive to rotor movements. The switching instant of a PWM cycle can often be approximated as a Dirac impulse. This Dirac impulse excites the resonant frequency of the eddy current sensor. The resonance then decays until the next switching instant. The quality factor is directly related to the decay time.

To amplify the excitation, the PWM switching could include a burst of transitions instead of a single transition. This could also shape the harmonics spectrum to concentrate the energy of the PWM harmonics to be close to the eddy current resonance.

By using the PWM frequency, harmonics thereof or PWM switching bursts, the coil resonance can be excited without an additional amplifier or, the need to isolate the amplifier from the motor driver with filter elements, reducing the size and increasing the reliability of the VAD control unit.

An additional or alternative measurement method to BEMF replication may be used. The following methods also enable detachment at standstill.

In a second method, the impedance of the actuator coils 3, which are motor coils, can, additionally or alternatively, be modified by coil impedance modulators 13. The coil impedance modulators 13 can be implemented as magnetically sensitive capacitors connected to the actuator coils 3, magnetically saturable components whose saturation due to the magnetic field generated by the rotor magnets change the reluctance of the magnetic flux circuit through the actuator coils 3 or, dedicated coils or an active electronic circuit using magnetic field sensors.

An additional force is acting on the rotor, if magnetically saturable components are used inside the stator. Then, a compromise between high sensor signal and low rotor forces has to be made by using an optimal material and amount of magnetically saturable material. A high frequency current has to be imprinted on the phase current to measure the phase impedance at that frequency. All methods have in common, that they modulate the impedance of the actuator coils 3 and therefore no dedicated wires, in addition to the motor wires, are used to read out their signal.

The impedance of the actuator coils 3 is measured in the control unit with an impedance analyser 14. The impedance analyser excites the resonance or another high frequency inside the actuator coils 3 and observes the phase current or terminal voltage to determine the impedance. This method is also limited by noise and benefits from a low noise motor driver and filtering of the switching noise. Alternatively, the motor driver switching action could excite the actuator coils 3 with an RF current or a harmonic component of the PWM. The impedance signal is fed to another rotor position estimator 7 and also provides a rotor position signal to the levitation controller.

A third method of rotor position measurements (not shown) uses dedicated sensors like, but not limited to, hall effect, eddy current sensors, fluxgate sensors or ultrasonic sensors to measure the rotor position. The sensor signal is imprinted on the driveline signals. The signals could be frequency, amplitude or code modulated prior to imprinting them onto the driveline. An RF-receiver, similar to the impedance analyser, detects the signals inside the control unit to provide a rotor position signal. In one embodiment, the actuator coils 3, which are motor coils, are used to oscillate the rotor in axial, radial, rotational or tilt direction at an audible or ultrasonic frequency. Microphones or the actuator coils 3 pick up the sound that originates from the rotor. The time delay or phase shift between acoustic sender and receiver can be used to measure the rotor position or volume flow through the VAD.

The levitation controller 8 uses one or multiple of the available rotor position signals to close the levitation control loop. Based on the motor and levitation operating the position measurements could be weighted or thrusted differently. To aid the weighting, the rotor position estimators 7 can optionally prove a signal quality indicator. If any of the signals deviate from another or a simulated pump model, an alarm or log entry can optionally be triggered.

The VAD system in FIG. 10 can be implemented by using any combination of blocks from FIG. 9 and FIG. 10. It is e.g. possible to use BEMF or impedance based measurements with a switching mode motor driver or, use only one of the provided methods for rotor position measurement.

The sensorless motor VAD as described in this application can also be combined with a dedicated bearing coil to either isolate the sensor and actuator signals from each other or, to increase the efficiency of the levitation control.

FIG. 19 shows an arrangement of motor coils that can be used to measure an axial and/or a rotational position of the rotor with eddy current measurements using the motor coils. The coils are arranged in a plane in a circular manner. In FIG. 19 only 3 out of 12 coils are depicted, wherein the 3 coils can be considered as being arranged on a quarter circle. FIG. 19 also depicts the impeller of the blood pump. The impeller may rotate in a plane parallel to the plane of the coils. During rotation, the inductance of each coil may vary in correspondence with the non-uniform structure of the impeller as depicted in FIG. 19. It is hence possible to estimate an angular position of the rotor from the coil impedances. The distance between the rotation plane of the impeller and the plane of the coils determines the coil impedances as well. Therefore, the coil impedance may also be used to estimate the axial impeller position, which is a function of the distance between the rotation plane of the impeller and the plane of the coils.

At a high frequency, the stray capacitance of the motor windings creates a resonant circuit (resonator) with the winding inductance. Most commonly, the eddy current sensors are operated close the self-resonant frequency of the sensing coil. The eddy current modifies the inductance value, so that the resonant frequency changes (FIG. 20, right panel: Rotor). Another option to modify the resonant frequency is to tune the stray capacitance. Because of the high frequency, only very small capacitances are necessary to shift the resonant frequency significantly with an additional tuning capacitor (FIG. 20, left panel: Stator).

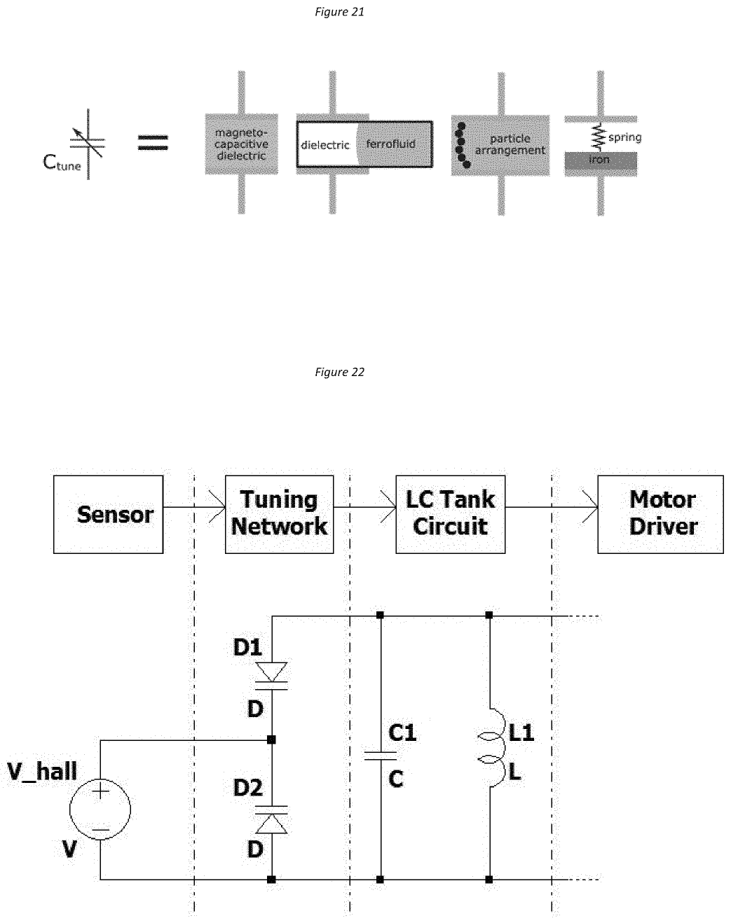

The tune capacitor would be built such that the capacitance changes, depending on the rotor position. Either magnetic field strength or BEMF could be measured. Possible implementations include capacitors filled with a magneto-capacitive dielectric (FIG. 21, left).

The tuning capacitor could, alternatively, be partially filled with ferrofluid to change its dielectric constant, when the ferrofluid is moving under the contacts (FIG. 21, centre left).

Alternatively, particles could be suspended in an emulsion between the capacitor plates (FIG. 21, centre right). The particles have magnetic properties so that they form chains along magnetic field lines. These chains that are oriented parallel and perpendicular to the capacitor plates, lead to different complex impedances of the capacitor. Therefore, the orientation of the magnetic field can be sensed.

Instead of changing the dielectric, the geometry of the tune capacitor could be modified by the magnetic field due to reluctance forces (FIG. 21, right).

With more space available, any electronic sensor can be used to tune a varactor diode with its output signal. The capacitance of the varactor diode could then tune the resonant frequency (FIG. 22). Possible sensors include magnetic field sensors, capacitive distance sensors, radio frequency (RF) transceivers, ultrasonic transceivers or dedicated BEMF sensing coils, as well as flow or pressure sensors. An active sensor circuit could be supplied with parasitic power from the motor-lines, requiring no extra driveline leads. The signal of an electrical field sensor or, another rotor distance sensor, can be read out through a motor coil impedance measurement.

A tuning network as shown in FIG. 22 is required to translate the electrical sensor signal into an impedance adjustment. The tuning network can be made of variable capacitance diodes (as shown in FIG. 22), metal —insulator-semiconductor capacitors with a voltage dependent capacitance, transistors which couple an extra impedance onto the LCR tank circuit or an active circuit made of operational amplifiers that emulates an additional inductor or capacitor coupled to the LCR tank circuit.

The mentioned coupling mechanism can also be used to modify a LCR tank circuit whose inductance is not the motor winding. A dedicated LCR series resonance tank circuit can also be used to amplify the influence of the tuning network. Preferably, the series resonance frequency of the LCR tank circuit is placed close to the motor coil tank circuit parallel resonance frequency or significantly higher.

The harmonic components of a PWM are placed at n*f_PWM, with n {1, 2, 3, 4, . . . }. That means that the switching noise is confined within certain areas of the spectrum. The noise level in between these areas can be much lower.

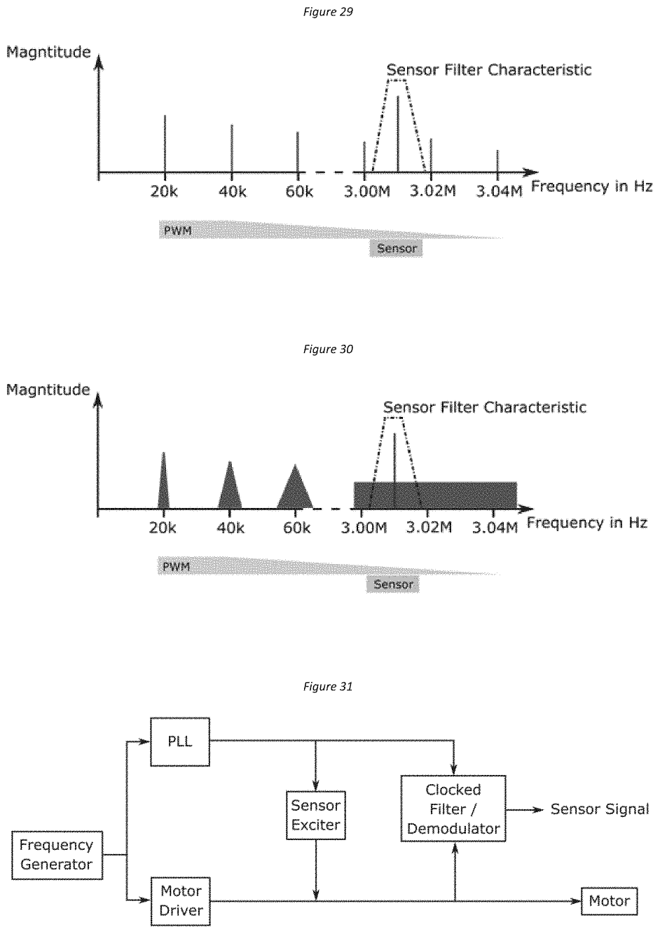

A narrow band RF sensor signal could be placed in such a low noise frequency range. However, the input filter of the sensor needs to be capable of suppressing the neighbouring PWM peaks (see FIG. 29). This frequency placement increases the requirements for the frequency stability of the PWM and the sensor signal.

FIG. 30 illustrates the origin of these requirements. If the PWM jitter at the fundamental PWM frequency (PWM period) is a certain amount, then the jitter at the n-th harmonics is n-times as wide. At high frequencies, the PWM spectrum merges into a continuous band without gaps. This is because the distance between harmonics is constant, but the jitter gets wider with higher frequencies, overlapping at some point. At this point, it is not possible to extract the sensor signal, without also picking up the switching noise.

Jitter is a mathematical method to describe frequency variations. Even with jitter, at any instant in time, the PWM spectrum consists of single frequency peaks and resembles the spectrum in FIG. 29. Only after averaging over time, the spectrum looks like displayed in FIG. 30. Because of this, the sensor frequency can always be placed in between the PWM harmonics. However, the sensor frequency and filter characteristic have to be adjustable.

The PWM frequency, sensor frequency and switching filter could be synchronized to the same clock source. This can keep the sensor frequency always between the PWM harmonics. The sensor filter could be realized, using a lock-in filter, which can easily be tuned with a clock source. Phase-locked-loops (PLL) and frequency dividers can be used to keep the PWM and sensor frequency at a certain ratio (see FIG. 31). Instead of the sensor frequency being clocked by a PLL, the motor driver could be synchronized to the sensor frequency. The important characteristic is that both are clocked directly or indirectly by the same frequency generator, so they see the same jitter. This reduces the requirements for additional filters and therefore safes space and increases reliability.

Now turning to safety aspects of the VAD.

The voltage and current signals at the output of the power filter 11 are mostly sinusoidal with the main frequency component at the electrical rotational speed of the motor. The output of the power filter is optionally connected to the motor with a corrosion tolerant connector 15, e.g. implemented as a direct feedback connector, which enables a measurement of the voltage at the motor terminals without the influence of connector contact resistance.

Power over motor lines, power line communication and motor line communication are methods, to read out and supply sensors inside a VAD without adding additional wires to the driveline. The methods disclosed in the patent application WO2018206754A1 can be used to transmit the signal of dedicated sensor signals from an implanted VAD to the controller. Possible sensors include, but are not limited to, rotor position sensors, acceleration sensors, gyroscopic sensors, blood flow sensors and blood pressure sensors.

Driveline defects and driveline infections make up a significant portion of VAD therapy failures. The common approach to reduce driveline defects is to use backup wires inside the driveline. If any wire breaks, then the corresponding backup wire can take over. An alarm usually informs the user about the state of emergency.

The common approach doubles the number of wires inside the driveline. A motor-only VAD like the HVAD has 6 instead of 3 wires in its driveline due to redundancy. This, however, increases the cross-section of the driveline and, therefore, increases the risk of driveline infections.

Other VAD systems line Heart Mate 3 reduce the number of driveline wires by supplying the pump with DC current and placing the motor driver or bearing driver inside the pump. With this approach, only one or two extra wires are necessary for a fail-safe driveline. The additional electronic components inside the pump increase its size and reduce the possible patient population.

This disclosure also refers to a fail-safe approach for external motor driver VAD systems.

The disclosed fail-safe approach utilizes a three phase BLDC motor. The driveline contains three wires which connect the motor phases to a phase leg each. The motor coils of the motor phases have to be connected in star (instead of delta) configuration. The star point is normally connected inside the motor and is not accessible from the outside. An additional backup-wire in the driveline connects the star-point to an additional backup phase leg inside the drive line (see FIG. 33).

The requirement for motor, bearing or sensor operation of a BLDC motor is that at least two of the motor windings must be supplied with an independent current. If, for example, in the non-redundant VAD system in FIG. 33 the coil L3 or the wire R3 or one of the switches M5 or M6 fails with an open connection, then the current in L3 is zero and cannot be set from the outside.

The remaining coils L1 and L2 are now connected in series and are therefore carrying the same amount of current. Only an oscillating, instead of a rotating, magnetic field can be created with this configuration and efficient motor operation is usually not possible. A reliable motor start is impossible with such a configuration.

By utilizing the additional driveline wire R4 and the additional phase legs M7 and M8, the currents of L1 and L2 are again independent of each other. This allows creating a rotating magnetic field and operation of the motor in a normal two phase fashion.

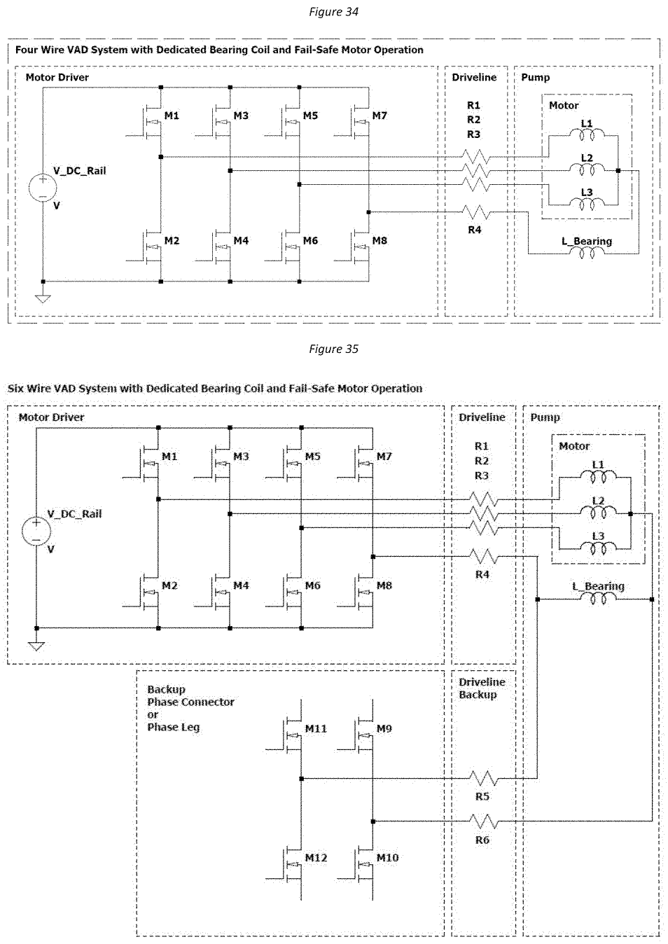

If a dedicated bearing coil is necessary, the bearing coil can be connected between the BLDC star point and a fourth driveline wire. This configuration, as shown in FIG. 34, can keep the motor in operation if any of the wires in the driveline, a motor coil or a phase leg fails with an open connection. The mayor drawback is that in the event of a failure the bearing can no longer be operated independently of the motor. This may be an option if the VAD has sophisticated backup bearings.

A major drawback of the presented configuration is that all of the bearing current passes through the motor wires, creating additional losses and magnetic fields. With zero force control the amount of bearing current should be small compared to the motor current, reducing the impact of the drawback.

With six wires the bearing and motor operation can be continued after an open connection failure in the driveline (see FIG. 35).

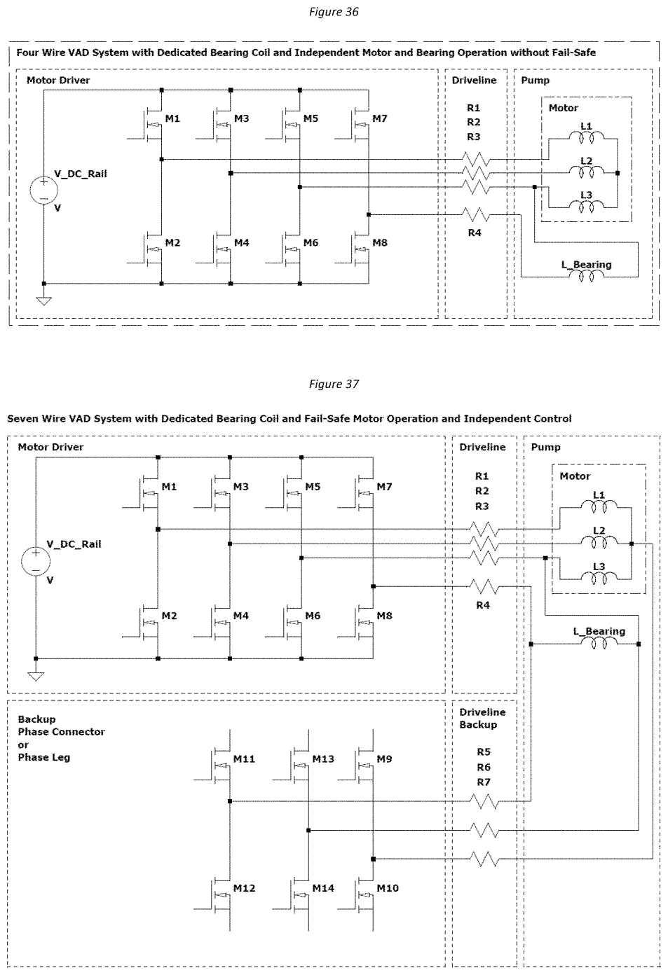

The four wire BLDC motor with dedicated active bearing configuration (FIG. 36) can be used when the risk to the patient due to a large driveline or an inefficient bearing is greater than the risk of an open connection failure.

The seven wire BLDC motor with dedicated bearing structure (FIG. 37) makes sure that even with an open connection failure the motor can be operated and the active bearing does not lose any efficiency.

For an accurate measurement of the BEMF as described above it can be advantageous to know the motor phase voltage accurately. Between the motor and the control unit, where the voltage can be measured, there are the driveline and the connector. Especially the connector suffers from gradual and sudden resistance variations. Movement of the contacts suddenly changes the contact point and contact pressure and therefore the contact resistance. The contacts within the connector are also much more exposed to the environment, than the wires within the driveline. This can lead to corrosion which gradually changes the contact resistance and also increases the sudden resistance variations due to a non-uniform corrosion.

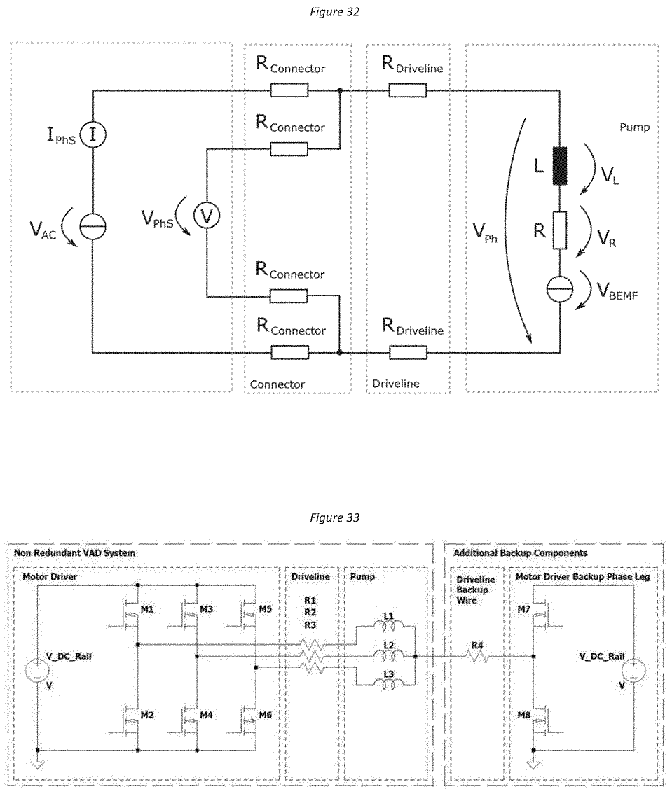

The proposed solution uses a connector with additional contacts to measure the phase voltage, without the influence of the connector resistance (see FIG. 32). The amount of wires in the driveline is not affected by the direct feedback connector.

One of the biggest contributions to noise originates from the switching events in the motor driver. The switching is clearly visible in the voltage and current waveforms (see for example FIG. 7).

So, switching noise from the motor driver can either be reduced or eliminated at the source by changing the motor driver, isolated from the measurement hardware with filters or, the measurement hardware or method could be made insensitive to the switching noise. The main noise source in a state of the art VAD motor driver (FIG. 9) is the switching mode motor driver 2.

As shown in FIG. 10, the switching noise can be significantly reduced with a switchless motor driver 10, which uses class AB power stages instead of class D PWM power stages, even though Class AB efficiency is lower than the efficiency of a PWM power stage. Class AB efficiency is 78.5% at its maximum at peak power output and reduces with lower output power.

By inserting an optional power modulator 9 between power source 1 and the switchless motor driver 10, the peak efficiency can be accomplished at any operating point. Alternatively, three (or more) power modulators, e.g. tracking DC-DC-converters or DC-AC-converters can be used as a low noise motor driver replacing the switchless motor driver 10 in FIG. 10. Optionally, the remaining noise left at the output terminals of the motor driver 10 is filtered with a three phase power filter 11, which suppresses the PWM frequency and its overtones in voltage and current.

The purpose of a motor driver is to generate a desired amount of torque in a motor. The torque is dependent only on the rotor position and the currents in the phase coils. The most common method to control the current is pulse width modulation (PWM). PWM switches the phase voltage rapidly between multiple voltage levels. Due to motor inductance, the current cannot follow the fast changes in applied voltage and creates a triangular current waveform. The exact amount of current is controlled with the PWM duty cycle. The current waveform is often smoothed using additional inductivities in series to the motor.

The major advantage of PWM is its energy efficiency. While one voltage level is applied to the phases, only very small resistive losses are created in the switching elements. Some additional losses are produced in the transition from low to high and high to low phase voltages. These losses can be reduced, by keeping the transition time as short as possible.

Small transition times are creating high frequency components. These frequency components can be an issue for electromagnetic emissions (EMI) and attached sensor devices. Common measures against high emissions are passive filters which either conduct the high frequency current components to ground or burn them as resistive losses. Small valued capacitors in combination with common mode chokes are usually sufficient to make a motor driver EMI compatible.

The high frequency components do not only radiate out of the driveline, they are also conducted to the sensor electronics attached to the motor driver. The most common form of sensor in motor drivers are phase current sensors. They are used to generate a current control feedback loop to accurately control the phase current. In sensorless BLDC motor drivers these current sensors are also used to estimate the rotor angle. Common rotor angle estimation methods include zero crossover detection, BEMF estimation or a model-based estimator approach.

To reduce the influence of fast switching times on the current measurement, it is quite common to synchronize the current sampling to the PWM switching, so that the time between sampling and switching is at a maximum or constant. It is also state of the art to use low pass filtering on the sensor signal, to suppress switching noise.

However, state of the art motor driver and current sensor concepts are neither sufficient for simultaneous motor operation and eddy current measurement, nor are they providing a high signal-to-noise current or voltage measurement that could be used to levitate a paediatric VAD rotor.

The BEMF can be estimated much more accurately in the absence of switching noise. BEMF detection, which is normally just sufficient for motor operation above a certain minimal motor speed, can also be used for fast rotor position measurement as described above.

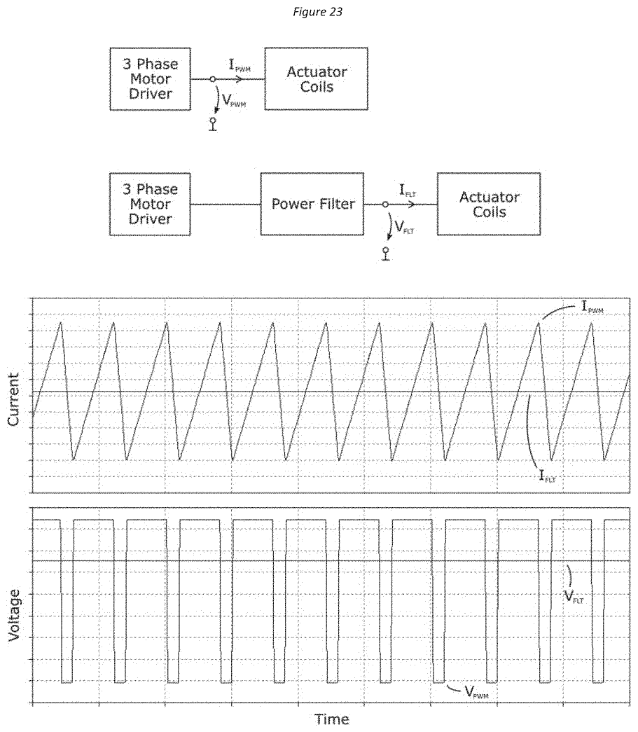

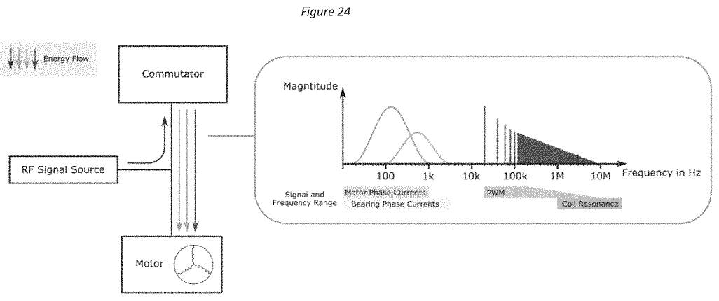

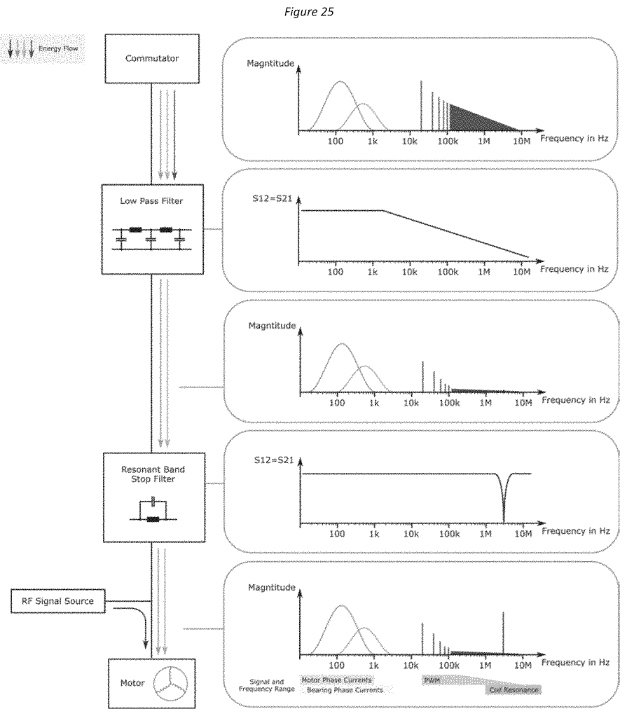

FIG. 25 shows the influence of an added power filter between motor driver and motor coils on the current and voltage waveforms. Here, a passive second-order filter was used to suppress harmonic components. Switching noise can be reduced to below 1%, compared to a setup without a power filter. This reduces the noise seen by sensing hardware that is also attached to the motor coils and therefore increases the signal-to-noise ratio.

The switching noise of a motor driver can be significantly reduced by not switching the output transistors at all. Instead, the output stage uses a class AB topology to linearly control the current in the motor phases. The drawback of a class AB output stage is its limited efficiency of maximum 78% at full output voltage swing. The efficiency is much lower if the maximum output voltage of the motor driver is much lower than the DC-rail voltage.

To keep the efficiency of the motor driver at an acceptable level, the DC rail voltage can be controlled with a DC/DC converter to be just above the maximum voltage swing (see FIG. 27).

Multiple Tracking DC/DC converters can be combined into an AC inverter (see FIG. 28). The AC inverter can directly control a motor phase current. The difference between a push-pull motor driver and an AC inverter or tracking dc controller is that the voltage waveform at the output of an AC inverter can be much smoother than the output from a PWM motor driver.

The reduced output switching noise of a motor driver with a “class AB” or “tracking DC/AC” topology reduces the high-frequency components in the motor phase currents and voltages. The absence of high-frequency components reduces the disturbances in a motor-based measurement system like e.g. BEMF or motor-coil eddy current sensors. The larger inherent signal-to-noise ratio increases the tolerance level of the VAD system to electromagnetically radiated or conducted interferences.

As described above, a high frequency current may be imprinted on the motor coils to perform eddy current measurements with the motor coils.

When connecting a high frequency source to the driveline (see FIG. 24), the RF energy will flow into the motor driver. This is due to the high impedance of the motor at motor coil resonance and a relatively low input impedance of the motor driver at the motor coil resonance frequency of several MHz. The load on the RF source is high, but no RF current flows in the motor coils. Because the motor coil resonance is not excited, the RF current has no dependency on the specific impedance characteristic of the motor coils. Therefore, no rotor position sensing is possible.

In order to direct the RF current to the motor, the RF input impedance of the motor driver must be high, at least at the resonance frequency of the motor coils. Resonant band stop filters and/or low-pass filters can be used to increase the impedance at a specific frequency (see FIG. 25). With this method a high filter quality can be achieved, with only two passive components per phase.

Now the RF current can excite the motor coil resonance. To measure the impedance accurately, no other RF source should excite the resonance.

However, the harmonic components of the PWM can reach up to several MHz. This can significantly reduce the signal-to-noise ratio of a sensitive rotor position measurement system.

To prevent the PWM voltage harmonics from being converted into harmonic motor currents, a low pass filter can be used (see FIG. 25). A wide range of frequencies (10 Hz-2 kHz) has to pass this filter while a broad spectrum of PWM harmonics need to be suppressed. Passive low-pass filters (with multiple stages) can at least reduce the harmonic PWM components.

More filter elements may be necessary to couple the RF source to the driveline or, to prevent the voltages on the driveline from damaging the RF source.

The common approach to suppress PWM noise on shunt current sensors is to synchronize the sampling to the PWM frequency.

The shunt signal is often also filtered using a low pass filter, for example a passive low pass filter. Such a filter can be made more effective than the filters in series to the motor phases since only a low sensing current instead of the motor phase current is entering the filter.

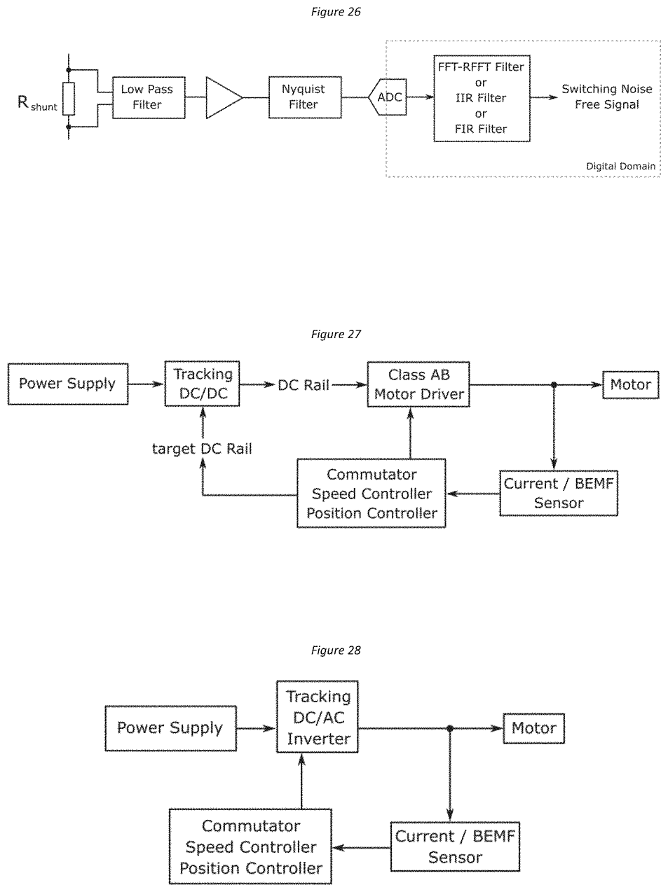

A greater attenuation of noise can be achieved with digital filtering (see FIG. 26). Frequency components of the signals to be attenuated must be within the Nyquist frequency range, that is, the frequency components are less than half of the sampling frequency. Therefore, the sample frequency must be significantly higher than the PWM frequency. In order to efficiently suppress signal frequencies above the Nyquist frequency range, a Nyquist filter may be applied before analog-digital conversion, wherein the Nyquist filter is configured to attenuate frequencies above the Nyquist frequency range.

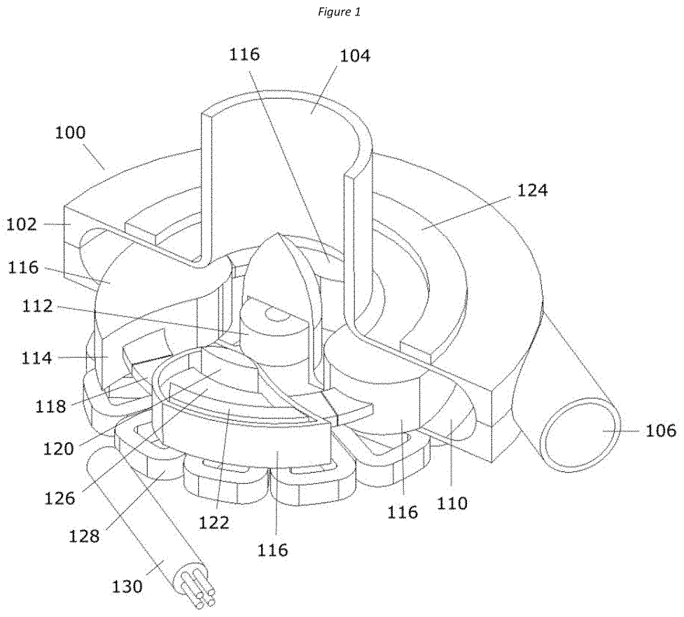

All the measurement methods concerning estimating the pump rotor position and orientation and, the safety-related technical aspects concerning operating a blood pump safely, outlined in the above sections are applicable to a variety of alternative pump designs. Various exemplary designs of a blood pump are outlined in the below sections, that is, to blood pumps according to FIG. 1 and FIG. 38 et seq. in particular.

FIG. 1 shows an exemplary blood pump. The blood pump 100 includes a housing 102 with an axial inlet 104, and an outlet chamber 106. The outlet chamber 106 includes an outlet 108, which can be connected to a graft or tube to be connected to the vascular system such as the artery. The inlet 104 can be inserted into the apex of a heart ventricle or, can also be attached to a graft or tube which is attached to the vascular system.

The outlet chamber 106, which may be designed as a volute, includes a back plate 110 away from the inlet. In the example shown, the back plate 110 includes a central spire which extends in the direction of the axial inlet and houses a permanent magnet 112. The chamber 106 houses a magnetically levitatable impeller 114, including four blades 116 (some are shown in a cut-through view to show the inside of the blade) which are connected to each other via webs 118. The impeller includes a plurality of permanent magnets: Each blade includes a permanent magnet 120 which acts as the counterpart to permanent magnet 112. This system of magnets is part of a passive radial magnetic bearing. The impeller further includes an optional tilt bearing magnet 122 (preferably also in each blade), which interacts with a magnetic ring 124 placed on the housing, thereby forming a tilt bearing.

Furthermore, each blade includes a rotor magnet 126, which interacts with the motor coils 128. The motor coils are placed on the far side of the back plate and are ironless, preferably copper windings. The power, i.e. current and voltage, within the motor coils is controlled via a control unit (not shown in FIG. 1) which is preferably placed outside of the human body. The coils and the control unit are connected via a drive line 130. The drive line 130 includes four wires, wherein one of the wires can be used as a redundant wire. The wires are used to control the motor coils such that the impeller can be rotated and the position of the rotor, at least in the axial direction, can be measured via one of the control schemes, methods or circuitry outlined in this application. Since the blood pump has no separate rotor position sensor, the wires needed within the drive line can be reduced compared to conventional blood pumps.

In other embodiments the pump may have further sensors; however, the motor coils are used for sensing and measuring a position of the rotor.

FIG. 38 shows a blood pump that can take advantage of the presented improved bearing concept. The blood pump 50 contains an inflow 58 and at least one outflow 59. A rotor 66 is driven by actuators comprising (or in some embodiments consisting of) rotor-magnets 51 and actuator-coils 52 and, additionally or alternatively actuator-coils (motor-coils) 69.

In operation, the rotor rotates mainly around the pump axis 67. The rotor 66 is magnetically levitated inside the blood pump 50. A passive magnetic bearing comprising at least two of the magnetic components 53, 54, 55 or 56 limits the radial movement of the rotor. The radial bearing is unstable in axial direction and can have an unstable equilibrium position. The actuator components 51, 52 or 69 can be used to control the axial rotor position to the unstable equilibrium position or another predefined axial rotor position. Tilting of the rotor around the tilting point 68 is limited by one or more passive magnetic bearings, comprising at least two of the magnetic components 60, 61, 62, 63 or 64. The tilting could alternatively or in addition be controlled using the actuators components 51, 52 or 69. The blood pump can contain a central hub or spire 57 to hold the radial bearing component 54 in place. The hub can be minimized or omitted, if the radial bearing does not rely on the magnetic component 54. The transition from the hub 57 to the back plate 65 can be implemented gradually to improve fluid flow and hemodynamics. The actuator coils 52 or 69 can be positioned near the outflow, near the inflow or at both locations.

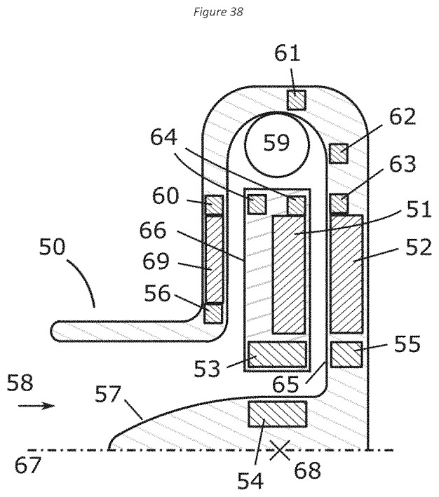

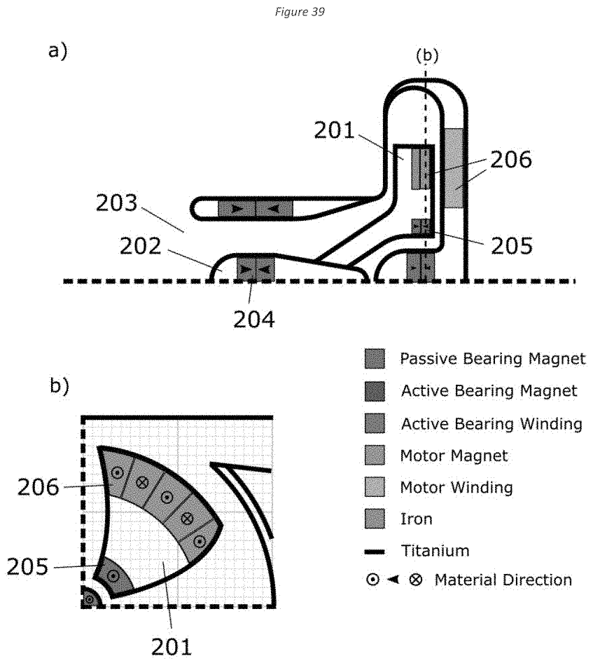

FIG. 39 shows another embodiment of a blood pump in a cross-section view (FIG. 39 a) and a top-view of a section of the rotor (FIG. 39 b). The rotor blades 201 are connected by a central rotating hub 202 that can extend into the inflow 203. The rotating hub contains a radial bearing 204 and the rotor blades are containing an additional passive magnetic radial bearing 205. Both passive magnetic bearings are stabilizing the rotor in radial direction and tilt direction. The unstable axial position is controlled by the actuator 206. It is possible that the magnets of the actuator may be arranged as a Halbach array. Halbach arrays may also be applied in subsequently described embodiments.

The blood pump according to FIG. 39 yields the advantage that a magnetic levitation of the pump rotor is accomplished without the need for complex pump-integrated electronics which, in turn, facilitates drivelines with a minimum of wires, e.g. three wires may be sufficient. In addition, said blood pump features an advantageously reduced design height, being one of the most critical design parameters in paediatric VADs. Also, a blood pump according to FIG. 39 promotes washout of thrombi from above and below the blades, for example, as compared to a shrouded pump design.

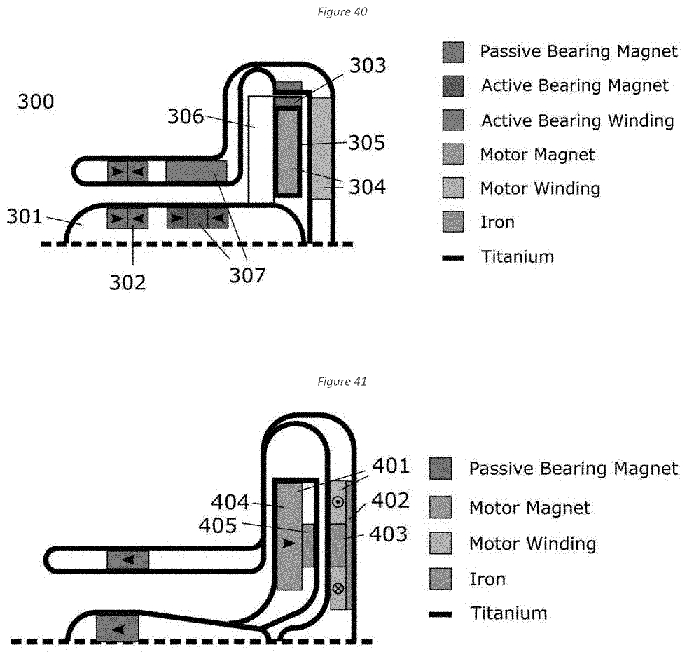

FIG. 40 shows a blood pump 300 with a central hub 301 and two axially separated passive magnetic bearings 302 and 303. The magnet components of a motor or actuator 304 are placed inside a disc 305 inside a volute near the outflow or inside the rotor blades 306. The magnet disc is connected to the central hub via pump blades 306. An active magnetic actuator 307, which may also be provided as a Halbach array, could also be placed in the inflow and central hub. To increase the efficiency of the pump, motor 304 and dedicated active magnetic bearing 307 could be used together to control the axial rotor position, and/or rotor speed and/or tilting motion.

The blood pump according to FIG. 40 yields the advantage that a high efficiency in terms of axial force and torque generation is obtained that facilitates stiffer magnetic bearings which, in turn, yields a higher robustness of the bearing against external forces and accelerations Imparted to the pump rotor, for example. Also, a high efficiency gives the advantage of a reduced heating of the blood pumped by through the pump which reduces the risk of formation of thrombi.

FIG. 41 shows a variation of the blood pump 300 shown in FIG. 40, where the radial magnetic bearing 302 inside the volute is omitted. The bearing function is now carried out by the motor 401, which contains a magnetically conductive back plate 402 or teeth 403 which are attracted by the rotor magnets 404. A magnetically conductive field concentrator 405 could be used to optimize the bearing capabilities of the motor bearing.

The blood pump according to FIG. 41 yields the advantage that a magnetic levitation of the pump rotor is accomplished without the need for complex pump-integrated electronics which, in turn, facilitates drivelines with a minimum of wires, e.g. three wires may be sufficient. Furthermore, due to the pump motor and the rear magnetic bearing being the same component, a simpler design of the blood pump according to FIG. 41 is gained.

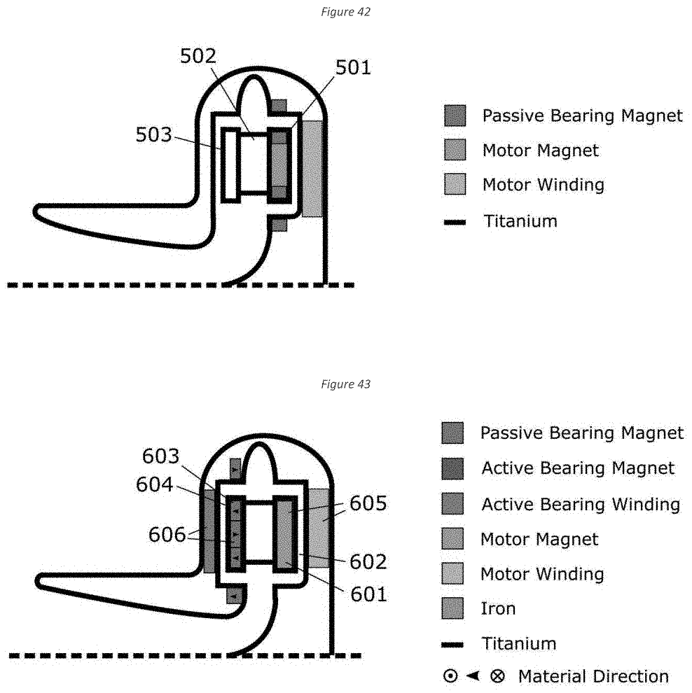

FIG. 42 shows a variation of the blood pump in FIG. 1 or 38. The magnetic components of the rotor are contained in a disc 501. The pump blades 502 are located on top of the rotor disc 501. A second disc 503 could be placed opposite to the first disc. The magnetic components could be located in either or both discs.

The blood pump according to FIG. 42 yields the advantage that a magnetic levitation of the pump rotor is accomplished without the need for complex pump-integrated electronics which, in turn, facilitates drivelines with a minimum of wires, e.g. three wires may be sufficient. Additionally, with a blood pump according to FIG. 42, a flexible inlet geometry is acquired that, in turn, yields diverse implantation positions for such a blood pump.

The blood pump in FIG. 43 shows one variation of the blood pump displayed in FIG. 42. The motor magnets 601 are located in one disc 602 and the bearing magnets 603 are located in the other disc 604. The field of the bearing magnets 603 can be used for an active axial bearing. Motor 605 and active axial magnetic bearing 606 could share the load of the rotor axial position control to increase maximum force and/or efficiency.

As with the blood pump according to FIG. 40, the blood pump according to FIG. 43 yields the advantage that a high efficiency in terms of axial force and torque generation is obtained that facilitates stiffer magnetic bearings which, in turn, yields a higher robustness of the bearing against external forces and accelerations imparted to the pump rotor, for example. Also, a high efficiency gives the advantage of a reduced heating of the blood pumped by through the pump which reduces the risk of formation of thrombi. Additionally, with a blood pump according to FIG. 43, a flexible inlet geometry is acquired that, in turn, yields diverse implantation positions for such a blood pump.

FIG. 44 shows the load sharing of the motor 701 and the active axial bearing 702, implemented in a shroudless rotor centrifugal pump 700. Motor 701 and active bearing 702 can be separated axially as shown or, separated radially. The blood pump according to FIG. 44 yields, in general, similar advantages to a blood pump according to FIG. 43.

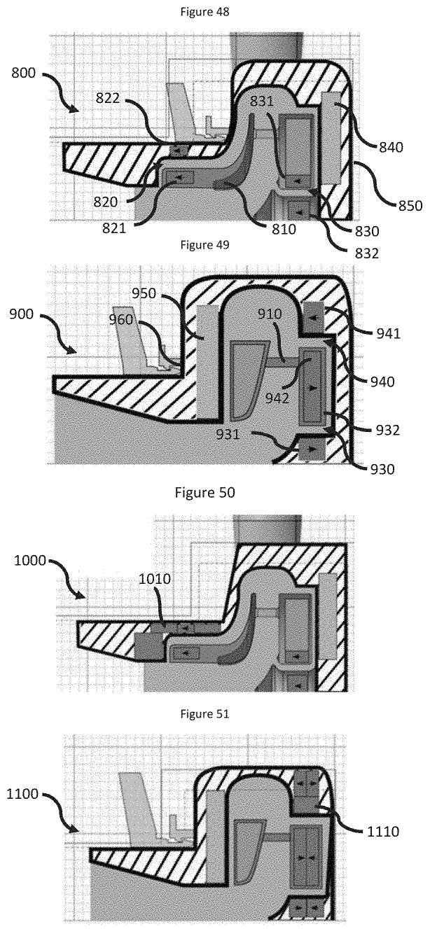

FIG. 48 shows another embodiment of a blood pump 800. This blood pump 800 is similar to the technical design of the blood pump shown in FIG. 39. The passive magnetic bearing of the pump rotor 810 is accomplished through two axially-displaced radial bearings 820, 830. Each of which exhibits a radially stabilising and an axially destabilising effect. In combination, these two axially displaced radial bearings 820, 830 yield a tilt stabilisation of the pump rotor.

Further, each passive radial bearing 820, 830 at hand consists of two radially repulsive magnetic elements 821, 822, 831, 832. In addition, the motor stator 840 is realised as an ironless axial-flux motor and, is disposed at the bottom of the pump casing 850.

The blood pump 900 in FIG. 49 shows a variation of the blood pump, displayed in FIG. 42. With this embodiment of a blood pump 900, the passive magnetic bearing of the pump rotor 910 is accomplished through two magnetic bearings which are arranged in one plane at the bottom of the pump casing 920.

A first magnetic bearing, located radially inward, is implemented as a radial bearing 930. This bearing 930 consists of two radially repulsive magnetic elements 931, 932, further yielding a radially stabilising, but an axially and tilt destabilising effect.

Additionally, a second magnetic bearing, located radially outward, is implemented as an axial, tilt-stabilising bearing 940 in turn. Here, the bearing consists of two radially attracting magnetic elements 941, 942, yielding an axially and tilt stabilising, but a radially destabilising effect now.

Tilt stabilisation of the pump rotor 910 is accomplished through the moment arm that originates from the radial location of the axial bearing 940, relative to the axis of rotation of the pump rotor 910. Here too, the motor stator 950 is realised as an ironless axial-flux motor and is now disposed at the opposite side of the pump casing, the pump lid 960.

The blood pump 1000 in FIG. 50 shows a variation of the blood pump, displayed in FIG. 48. With this embodiment of a blood pump, the passive magnetic bearing of the pump rotor is accomplished in an identical fashion to the embodiment of a blood pump shown in FIG. 48.

With the blood pump of FIG. 50, however, the axial positioning of the pump rotor is accomplished by means of an active bearing winding 1010 alone, disposed at the inlet of the pump. During pump operation, the axial-flux motor here merely generates torque, to rotate the pump rotor.

The blood pump 1100 in FIG. 51 shows a variation of the blood pump, displayed in FIG. 49. With this embodiment of a blood pump, the passive magnetic bearing of the pump rotor is accomplished in an identical fashion to the embodiment of a blood pump shown in FIG. 49.

With the blood pump of FIG. 51, however, the axial positioning of the pump rotor is accomplished by means of an active bearing winding 1110 alone, now disposed at the bottom of the pump casing. During pump operation, the axial-flux motor here merely generates torque, to rotate the pump rotor.

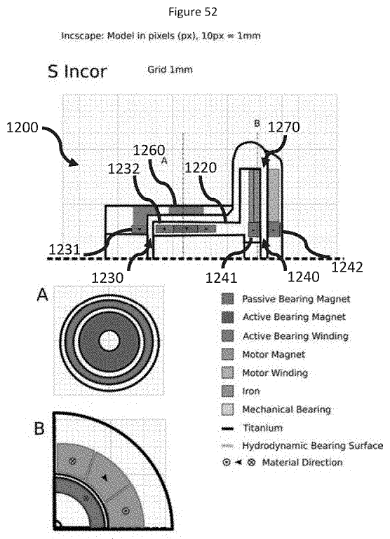

FIG. 52 shows another embodiment of a blood pump 1200. This blood pump 1200 is similar to the technical design of the blood pump shown in FIG. 48.

The passive magnetic bearing of the pump rotor 1220 is accomplished through two axially-displaced axial bearings 1230, 1240. Each of which exhibits a radially stabilising and an axially destabilising effect. In combination, these two axially displaced axial bearings 1230, 1240 yield a tilt stabilisation of the pump rotor. Further, each passive radial bearing 1230, 1240 at hand consists of two axially attractive magnetic elements 1231, 1232, 1241, 1242.

In addition, with the passive magnetic elements, associated with the pump motor 1270 and the passive magnetic bearing, a Halbach configuration is realised. This yields the advantage of a maximum of controllability of the pump rotor which, in turn, yields a higher robustness of the bearing against external forces and accelerations imparted to the pump rotor, for example.

Further, with the blood pump of FIG. 52, the axial positioning of the pump rotor is accomplished by means of an active bearing winding 1260 alone, disposed at the inlet of the pump. During pump operation, the pump motor here merely generates torque, to rotate the pump rotor 1220. Alternatively or additionally, in another embodiment, control of the axial position of the pump rotor 1220 is accomplished by means of a combination of magnetic bearing forces and axial motor forces which, in turn, yields the highest total axial stability of the system, i.e. of the pump rotor 1220.

Hence, by means of the active bearing winding 1260, in combination with the Halbach magnets and, optionally, in combination with the axial motor force, a high operational efficiency is obtained, that gives the advantage of a reduced heating of the blood pumped by through the pump which reduces the risk of formation of thrombi, for example.

The Blood pump system may also include a connection system for use in medical applications comprising a cannula c7 made of a flexible material, a claw ring c1 disposed on the cannula c7 and having at least two claws c11, wherein the claw ring c1 encompasses an outer surface of the cannula c7 and is arranged on a cannula end c71 of the cannula c7 for rotation and axial displacement on the cannula c7 to a stop, the stop comprising a collar on the cannula end c71 on the outer surface of the cannula c7, and a tube c5 comprising a locking ring c3 attached to a tube end and a nipple attached to the tube c5, wherein the claw ring c1 is capable to be joined with the locking ring c3 by an axial movement of the claw ring c1 with respect to the cannula c7 towards the locking ring c3 and by latching of the at least two claws c11 on the locking ring c3 in a position in which this axial movement is limited by the stop.

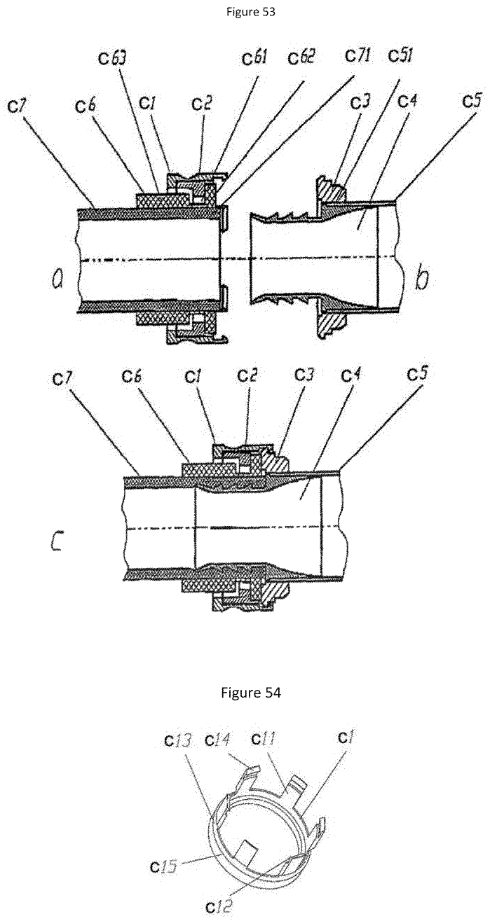

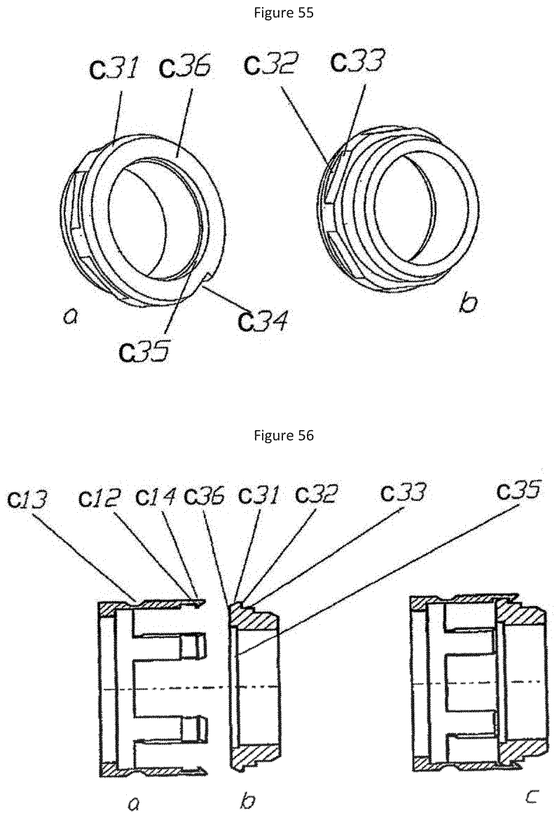

FIGS. 53 to 57 show an exemplary device of the connection system for use in medical applications which provides a connection between a cannula c7 and a tube c5. A nipple, shown here as a hose coupling c4, is inserted in a cannula end c71 (see FIG. 53b). A reinforcement element c6 is disposed on the cannula end c71 and fixedly connected with the cannula end c71. A spacer ring c2 is arranged in a groove c61 of the reinforcement element c6. A claw ring c1 is latched with its claws c11 on the locking ring c3, with the claws c11 of the claw ring c1 being attached to a base ring c15 (see FIG. 54). The claw ring c1 presses hereby on the spacer ring c2 which in turn presses on a collar c62 of the reinforcement element c6, thereby forming a tight connection with an annular end face c36 of the locking ring c3. The seal between the cannula c7 and the hose coupling c4 is formed by an elastic expansion of the cannula end c71, when the cannula end c71 is pushed onto the hose coupling c4. This produces radial sealing forces that press the inner surface of the cannula end c71 onto the hose coupling c4. The radial sealing force is further increased by the tension ring c63 of the reinforcement element c6. The device is essentially a snap connection. The claws c11 disposed on the base ring c15 are pushed over the locking ring c3, where they releasably latch.

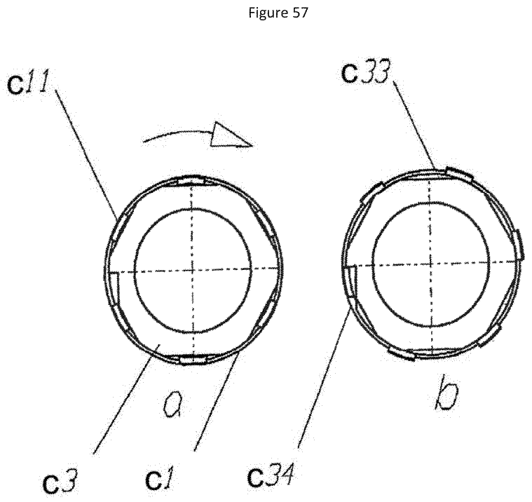

The end face c36 transitions radially outwardly into a sloped face c31 and radially inwardly into a clearance c35, which receives the cannula shoulder disposed before the collar and produced when the cannula is shortened during implantation. Holding faces c32 and sloped faces c33 are disposed behind the end face c36, with the claws c11 of the claw ring c1 disposed on the cannula snapping into the faces c32 and c33, thereby connecting the cannula c7 with the tube c5. The spacer ring c2 and the claw ring c1 for connecting the cannula c7 can already be pre-mounted on the cannula c7 when the cannula is supplied or can alternatively be mounted on the cannula c7 during surgery. For making the connection, the cannula c7 is pushed onto the hose coupling c4, thereby elastically widening the inside diameter of the cannula c7. The claw ring c1 is rotated until it is positioned relative to the locking ring c3 in the latching position (FIG. 57a), and is then axially displaced towards the locking ring c3. The claws c11 are spread apart when pushed onto the sloped faces c31 of the locking ring and latch with claws support surfaces c12 on the locking ring holding surfaces c32 (FIG. 56a, 56b, 56c). In a perspective illustration of the claw ring c1 according to FIG. 54, six claws c11 are arranged on a base ring c15. The claw holding surface c12 and a sloped claw surface c14 are disposed on the ends of the claws c11. These are provided to enable a safe latching engagement with the locking ring c3, as illustrated in FIG. 56a, 56b, 56c. Resilient joints c13 on the claw ring c1 allow the claws c11 to spread when they snappingly engage with the locking ring c3.

The blood pump system may also include a device for connecting a cannula with a hollow organ, in particular with a heart, wherein a cannula tip of the cannula has an opening which, for the prevention of complete occlusion and retention of blood flow from the hollow organ into the cannula, is waved at its upper edge and provided with recesses.

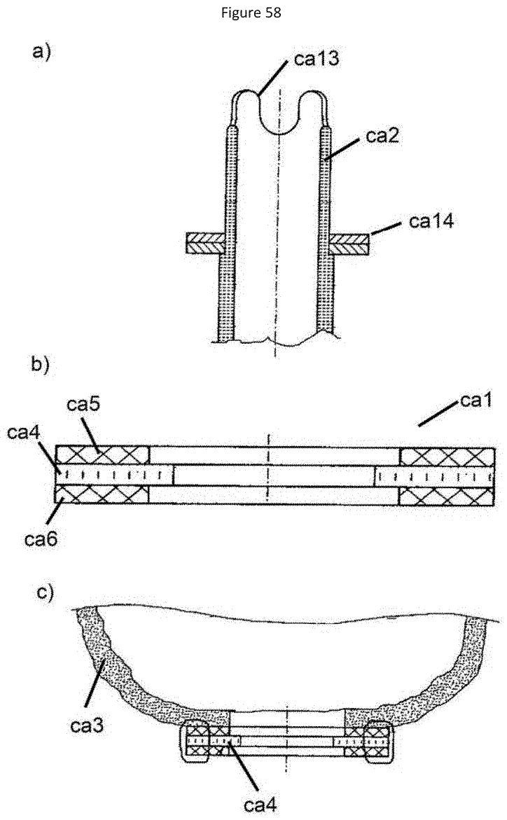

In an exemplary embodiment of the device for connecting the cannula with the hollow organ shown in FIG. 58, the upper edge of the cannula tip ca13, which may protrude into a left ventricle of a heart ca3, is wavy shaped and provided with deep, semi-circular recesses. In this case, the recesses prevent complete occlusion of the opening, so that blood can continue to flow from the ventricle into the inlet cannula.

The cannula may be combined with a suture ring ca1 suturable at the heart ca3. For example, to connect the inlet cannula with the left ventricle ca3, a circular opening is first cut out at the apex of the heart into which the cannula tip ca13 will be inserted later. Before inserting the cannula tip ca13, a suture ring ca1 is sutured around said circular opening.

The cannula may have a suture flange ca14. In an example, the suture ring ca13 has the same diameter dimensions as a suture flange ca14 at the inlet cannula cat. In an example, the suture ring ca1 may consist of a five layer silicone core, which consists of an inner layer ca4 of unreinforced silicone and layers ca5, ca6 of reinforced silicone on both sides; both, on the top and on the bottom a Dacron velour fabric is glued.

All shown pump setups (FIG. 1, 38-44, 48-52) are designed for, and compatible with, levitation via BEMF based rotor position detection and motor coil impedance based rotor position detection, enabling sensor redundancy without additional implanted components. By utilizing the axial forces generated by an axial flux motor, a dedicated bearing coil can be minimized or completely omitted, reducing the pump size further. The disclosed pumps do not need active levitation electronics inside the pump and are therefore keeping the size small and reliability high. Some reliability measures, e. g. a fully redundant dc-link to the pump, cannot be accomplished without pump electronics. Therefore, alternative measures were disclosed which increase the redundancy and are compatible to the disclosed levitation structure. When optimizing the pump size, it is important to also keep the implanted peripherals as small as possible without compromising hemocompatibility. The disclosed connection system is therefore especially suitable for the disclosed pump setups.

The disclosed pump systems combine small size, reliability and hemocompatibility, which makes them especially suitable for a paediatric VAD.

The control unit may include a controller or any other processor. The processor may be one or more devices operable to execute logic. The logic may include computer executable instructions or computer code embodied in memory of the processor, in memory of the control unit, and/or in any other memory that when executed by the processor, cause the processor to perform the features implemented by the logic. The computer code may include instructions executable with the processor.

To clarify the use of and to hereby provide notice to the public, the phrases “at least one of <A>, <B>, . . . and <N>” or “at least one of <A>, <B>, . . . or <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

1. A blood pump system comprising:
a blood pump,
a drive line, and
a control unit for controlling operation of the pump,
the pump comprising:
a housing, including an inlet and an outlet
a motor actuator, wherein the motor includes a plurality of motor coils for driving an impeller; and
a rotor including the impeller, wherein the impeller is located in the housing and includes a plurality of rotor magnets;
wherein the control unit is configured to:
operate the motor, such that the impeller rotates around an axis; and
measure the rotor position in a direction along the axis using at least one of the plurality of the motor coils.

2. The blood pump system of claim 1, wherein the control unit is configured to reduce or eliminate switching noise from a motor driver.

3. The blood pump system of claim 1, wherein an output stage of the motor driver includes filter elements for filtering out high frequency signals.

4. The blood pump system of claim 3, wherein a high frequency signal is added to the filtered motor driver output.

5. The blood pump system of claim 1, wherein measurement of the motor currents includes a measurement of the motor coil impedance, preferably the high frequency motor coil impedance.

6. The blood pump system of claim 1, wherein a motor internal back-electromotive force is replicated outside the motor, preferably using inductive shunt voltage measurement.

7. The blood pump system of claim 1, wherein a magnetic field strength is replicated in an electrical or digital signal outside the motor, preferably using a back-electromotive force replica and a matched pair of high-pass and low-pass filter elements.

8. The blood pump system of claim 1, wherein the control unit is configured to reduce voltage transients in the driveline or is configured to reduce trapezoidal or triangular current wave-forms with respect to the sinusoidal current waveforms.

9. The blood pump system of claim 1, wherein the control unit includes a DC-DC converter, or includes class AB amplifiers, and/or passive filter elements.

10. The blood pump system of claim 1, wherein the driveline includes no more than four wires, preferably three wires and one redundant wire.

11. The blood pump system of claim 1, wherein the blood pump includes a passive magnetic radial bearing and/or a passive magnetic tilt bearing.

12. The blood pump system of claim 1, wherein the blood pump includes an active axial magnetic bearing.

13. The blood pump system of claim 1, wherein the motor is an axial flux motor, preferably an ironless axial flux motor.

14. The blood pump system of claim 1, including a capacitor electrically parallel connected to a motor coil, wherein the motor coil and the capacitor form a resonant circuit having a resonance frequency and an electrical impedance with a magnitude and a phase.

15. The blood pump system of claim 14, wherein the motor coil includes a first coil and wherein a first capacitor is electrically parallel connected to the first coil and both forming a first resonant circuit, and wherein the motor coil includes a second coil and wherein a second capacitor is electrically parallel connected to the second coil and both forming a second resonant circuit, wherein a capacitance of the first capacitor is different from a capacitance of the second capacitor and the resonance frequency of the first resonant circuit is different from the resonance frequency of the second resonant circuit.

16. The blood pump system of claim 15, further including a measurement unit configured to determine the electrical impedance of one or more of the resonant circuits.

17. The blood pump system of claim 16, further including an estimation unit configured to estimate a translational and/or a rotational position of the rotor based on the electrical impedance of one or more of the resonant circuits.

18. The blood pump system of claim 1, wherein a test signal is fed into a motor coil, wherein the test signal includes a component which is at least one of amplitude modulated, frequency modulated, phase modulated, code modulated, wherein the code modulated component preferably includes a random code modulated component or a pseudo random code modulated component.

19. The blood pump system of claim 18, further comprising a detector unit, preferably including a correlator or a synchronous detector, configured to detect the test signal in a voltage measured across the motor coil and/or in a signal derived thereof.

20. The blood pump system of claim 19, wherein the detector unit is configured to estimate the motor coil impedance based on the detected test signal.

21. The blood pump system of claim 20, wherein the motor coil impedance is continuously estimated during operation of the blood pump system.

22. The blood pump system of claim 20, wherein the back-electromotive force replica is calculated with the estimated motor coil impedance.

23. The blood pump system of claim 21, wherein the estimated motor coil impedance is estimated by minimizing the high frequency signal component within the back-electromotive force replica.

24. The blood pump system of claim 1, wherein a magnetic field strength is replicated in an electrical or digital signal outside the motor, preferably by integrating a back-electromotive force replica with an integrator, wherein the integrator is numerically stabilized by feeding back an output signal of the integrator via a moving average filter, which produces an averaged signal, to an input of the integrator.

25. The blood pump system of claim 24, wherein the averaging time of the moving average filter is one rotation period or an integer multiple of one rotation period of the rotor.

26. The blood pump system of claim 24, wherein the back-electromotive force replica is an input signal of the integrator and wherein the averaged signal is subtracted from the input signal of the integrator.

27. The blood pump system of claim 26, wherein the averaged signal is low pass filtered before being subtracted from the input signal of the integrator.

28. The blood pump system of claim 1, further including a connection system for use in medical applications comprising:
a cannula made of a flexible material, a claw ring disposed on the cannula and having at least two claws, wherein the claw ring encompasses an outer surface of the cannula and is arranged on a cannula end of the cannula for rotation and axial displacement on the cannula to a stop, the stop comprising a collar on the cannula end on the outer surface of the cannula; and
a tube comprising a locking ring attached to a tube end and a nipple attached to the tube, wherein the claw ring is capable to be joined with the locking ring by an axial movement of the claw ring with respect to the cannula towards the locking ring and by latching of the at least two claws on the locking ring in a position in which this axial movement is limited by the stop.

29. The blood pump system of claim 1, further including a device for connecting a cannula (ca2) with a hollow organ (ca3), in particular with a heart (ca3), characterized in that a cannula tip (ca13) of the cannula (ca2) has an opening which, for the prevention of complete occlusion and retention of blood flow from the hollow organ (ca3) into the cannula (ca2), is waved at its upper edge and provided with recesses.

30. The blood pump system of claim 29, wherein the cannula (ca2) is combined with a suture ring (ca1) suturable at the heart (ca3).

31. The blood pump system of claim 29, wherein the cannula (ca2) has a suture flange (ca14).
